(19) United States
(12) Patent Application Publication
(10) Pub. No.: US 2026/0026756 A1
(43) Pub. Date: Jan. 29, 2026

(54) PREDICTING A CARDIAC INDEX FOR A PATIENT

(71) Applicant: THE JOHNS HOPKINS UNIVERSITY, Baltimore, MD (US)

(72) Inventors: Danielle Gottlieb SEN, Baltimore, MD (US); Ignacio ALBERT SMET, Baltimore, MD (US); Cedric MANLHIOT, Baltimore, MD (US); Bhargava Kumar CHINNI, Baltimore, MD (US); Summer DUFFY, Baltimore, MD (US)

(21) Appl. No.: 18/997,841

(22) PCT Filed: Aug. 23, 2023
(86) PCT No.: PCT/US2023/030885

§ 371 Date: Jan. 23, 2025

Related U.S. Application Data
(60) Provisional application No. 63/373,464, filed on Aug. 25, 2022.

(51) Int. Cl. A61B 5/00; A61B 5/02; A61B 5/0205

(52) U.S. Cl. CPC A61B 5/7275; A61B 5/02028; A61B 5/02055; A61B 5/6808; A61B 5/7267; A61B 5/742

(57) ABSTRACT

A method for predicting a cardiac index (CI) for a patient includes receiving a set of a measurements from the patient that characterizes an anatomical condition, a physical condition, a physiological condition, or a combination thereof. The method also includes receiving a non-invasive physiological measurement from the patient. The method also includes performing feature extraction on the non-invasive physiological measurement to produce one or more extracted features. The method also includes combining the set of measurements and the extracted features to produce a dataframe. The method also includes predicting the CI for the patient. The CI is predicted using a prediction model based upon the dataframe.

### CROSS-REFERENCE TO RELATED APPLICATIONS

This application is the national stage entry of International Patent Application No. PCT/US2023/030885, filed on Aug. 23, 2023, and published as WO 2024/044225 A1 on Feb. 29, 2024, which claims the benefit of U.S. Provisional Patent Application No. 63/373,464, filed on Aug. 25, 2022, which are hereby incorporated by reference in their entireties.

### FIELD

The present disclosure relates to a system and method for predicting a cardiac index (CI) in a patient. More particularly, the present disclosure relates to a system and method for predicting cardiac indexes (CIs) for a plurality of patients based upon non-invasive physiological measurements from the patients.

### BACKGROUND

The vital signs of infants may be monitored to assess their state of health and wellbeing. This is particularly useful for children with congenital heart disease (CHD), which affects nearly 1% of all annual births in the United States. Neonate and infant patients with CHD suffer from one or more birth defects that affect the normal way the heart works. Their complex abnormalities in cardiac anatomy and physiology require close monitoring of their vital signs.

Vital signs used by cardiologists and cardiac surgeons to assess the functioning of the heart may include cardiac output (CO), as well as related parameters such as stroke volume (SV) and ejection fraction (EF). Cardiac output describes the amount of blood volume the heart pumps per unit time, and indicates how efficiently a patient's heart pumps blood through their arterial tree. Typical values are about 5 L/min in adults and about 250 mL/min/kg in neonates. When abnormally low, CO is associated with significant morbidity and mortality. Cardiac output is the product of stroke volume and heart rate:

$$CO = stroke volume ( SV ) \times heart rate ( HR ) \qquad ( 1 )$$

Stroke volume (SV) is the amount of blood pumped with each heartbeat (i.e., the mathematical difference between left ventricular end-diastolic volume (EDV) and end-systolic volume (ESV)):

$$SV = End Diastolic Volume ( EDV ) - End Systolic Volume ( ESV ) \qquad ( 2 )$$

Stroke volume represents the volume of blood ejected by the left ventricle with each heartbeat. Typical values are about 80 mL in adults and about 1.77 mL/kg in neonates.

Besides HR, CO, and SV, another vital sign used to assess cardiac function is the ejection fraction (EF), which indicates how efficiently the ventricle is emptying itself with each heartbeat.

$$EF = SV / EDV \qquad ( 3 )$$

EF relates to EDV and ESV as described in Equation 3, with typical values for healthy adults being about 50-65%, and an ejection fraction of less than 40% indicating systolic heart failure. For healthy children, typical values are about 56-78%, with left ventricular systolic dysfunction defined by an ejection fraction under 55%.

Current techniques to measure CO, SV, and EF in infants are thermodilution and the Fick oxygen principle (Fick), performed in the catheterization lab. Both techniques are invasive and require anesthesia, posing non-trivial procedural risks to the patient. Furthermore, these techniques only capture a single discrete time point measurement. Healthcare providers are unable to continuously track CO over time, almost certainly missing dynamic and clinically important changes. Neonates and infants with CHD present with complex underlying pathologies, often with dynamic changes after an operation, thereby rendering continuous monitoring of vital signs important.

Besides ECG monitoring, measuring CO and SV in a continuous, non-invasive manner with high clinical accuracy has often been considered a “holy grail” of cardiac monitoring. Most existing techniques in this field require in-dwelling catheters, which in turn can lead to complications with the patient, are inherently inaccurate in the critically ill, and require a specially trained operator. No wearable device exists to measure CO, SV, and EF continuously and non-invasively in pediatric patients or patients with CHD.

Conventional infant monitoring systems typically include a crib-side camera and microphone for capturing images and sounds generated by the infant, and a 1-way wireless system for transmitting these images to a remote display that can be viewed by a family member (e.g., a parent). With such a system, the parent can be removed from the crib and still determine whether the infant is sleeping, crying, or moving about. Typically, such systems include viewing devices that are custom-made, hand-held, and feature a simple display for rendering images of the infant and a speaker system for projecting their sounds.

Newer devices may also include monitoring of clinical parameters such as heat rate (HR), blood oxygen level (SpO2), and movement. An example of such a device is the Owlet smart sock and baby monitor. This infant monitor includes a wireless sensory sock that measures photoplethysmography (PPG) and accelerometer signals and processes them to derive HR, SpO2, and movement. Some versions also include a camera and a microphone. The app allows the user to view these data in real time.

Vital signs, such as heart rate (HR), respiration rate (RR), and oxygen saturation (SpO2) are sometimes measured from an infant in a hospital or medical clinic. A vital sign monitor, typically featuring a form factor similar to that of a desktop computer, measures an electrocardiogram (ECG) from the infant to determine HR. Such a measurement requires attaching disposable adhesive electrodes to the infant's torso, and then connecting these to an ECG system within the vital signs monitor using a collection of electrical leads. The monitor can also measure RR with a technology called impedance pneumography (IP) which relies on the same electrodes used for ECG-based measurements of HR. In IP measurements, one electrode typically injects a low-amperage (e.g., 1 mA) current modulated at a high frequency (e.g., 50 kHz). Breathing-induced impedance changes in the infant's thorax create a measurable voltage change when combined with the injected current. The voltage signal can then be analyzed with signal-processing algorithms to determine RR. The monitor can also measure oxygen saturation using a pulse oximeter, generally placed in an area of high vasculature (e.g., fingers, toes). A pulse oximeter has a light source with at least two light sources in the red/infrared range (e.g., 660 nm and 900 nm) and a photodetector used to measure optical changes that correspond to changes in blood oxygen concentration, from which the SpO2 value is derived as a percentage.

Other vital signs, such as regional tissue oxygenation (regSpO2), are sometimes monitored during surgery or in the NICU to detect ischemia. The parameter regSpO2 is measured using near-infrared spectroscopy (NIRS), e.g., the INVOS™ 5100C.

Typically, HR, RR, SpO2, and other vital signs are measured from an infant in a neo-natal intensive care unit (NICU), pediatric intensive care unit (PICU), or similar (e.g., pediatric cardiac intensive care unit, PCICU).

Vital signs can also be monitored from the infant outside of the ICU and/or NICU (e.g., during a medical check-up). However, during such visits, infants tend to move and squirm about, making it difficult to measure vital signs such as HR, RR, and SpO2. In some cases, physicians can prescribe ECG monitors to ambulatory patients inside or outside a medical facility. These portable devices can measure and record time-dependent waveform signals to characterize ambulatory patients over short periods (e.g., 24-48 hours) using “Holter” monitors, or over longer periods (e.g., 1-3 weeks) using cardiac event monitors.

Conventional Holter or cardiac event monitors typically include a collection of chest-worn ECG electrodes (typically 3 or 5), an ECG circuit that collects analog signals from the ECG electrodes and converts these into multi-lead ECG waveforms. A processing unit then analyzes the ECG waveforms to determine cardiac information. Typically, the patient wears the entire system on his/her body. Some modern ECG-monitoring systems include wireless capabilities that transmit ECG waveforms and other numerical data through a cellular interface to an Internet-based system, where they are further analyzed to generate, for example, reports describing the patient's cardiac rhythm. In less sophisticated systems, the ECG-monitoring system is worn by the patient, and then returned to a company that downloads all relevant information into a computer, which then analyzes it to generate the report. The report, for example, may be imported into the patient's electronic medical record (EMR). The EMR avails the report to cardiologists or other clinicians, who then use it to help characterize the patient.

Finally, most infants wear diapers that collect urine and fecal matter, with a typical infant using as many as 5-10 diapers every day. Reusable diapers are typically composed of cloth materials, whereas disposable diapers are typically composed of a combination of plastic and cotton-like materials that collect and absorb the infant's waste. Disposable diapers come in many forms, but in general the American market is dominated by the Huggies and Pampers brands, which are developed and marketed by, respectively, Kimberly-Clark and Proctor and Gamble. In total, about 2 billion disposable diapers are deposited in America's landfills each year.

In addition, adult patients who suffer from incontinence may wear diapers. These patients are typically of old age and may also suffer from heart failure, chronic obstructive pulmonary disease, and other cardiac and respiratory diseases that benefit from improved and continuous monitoring of their health.

### SUMMARY

A system for monitoring a patient is disclosed. The system includes one or more drive electrodes configured to contact a skin of the patient and to inject an electrical current into the patient. The system also includes one or more sense electrodes configured to contact the skin of the patient and to measure a first signal from the patient that is not in response to the electrical current, and measure a second signal from the patient that is in response to the electrical current. The system also includes a control module configured to determine an electrocardiogram (ECG) waveform based at least partially upon the first signal, determine a bioimpedance waveform based at least partially upon the second signal, and determine a cardiac output of the patient based at least partially upon the ECG waveform and the bioimpedance waveform.

In another embodiment, the system includes one or more drive electrodes configured to contact a skin of the patient and to inject an electrical current into the patient. The electrical current includes an alternating current having a frequency from about 20,000 Hz to about 100,000 Hz. The system also includes one or more sense electrodes configured to contact the skin of the patient and to measure a first signal from the patient that is not in response to the electrical current, and measure a second signal from the patient that is in response to the electrical current. The system also includes a first light emitter configured to emit light toward the skin of the patient, and a second light emitter configured to emit light toward the skin of the patient. The light that is emitted from the first light emitter has a different wavelength than the light that is emitted from the second light emitter. The wavelengths of the light from the first and second light emitters are between about 500 nm and about 1000 nm. The system also includes a first photodetector configured to measure a third signal that is in response to the light from the first light emitter reflecting off of the skin of the patient. The third signal includes an optical signal. The system also includes a second photodetector configured to measure a fourth signal that is in response to the light from the second light emitter reflecting off of the skin of the patient. The fourth signal includes another optical signal. A distance between the first light emitter and the first photodetector is different than a distance between the second light emitter and the second photodetector. The system also includes a control module configured to determine an electrocardiogram (ECG) waveform based at least partially upon the first signal. The control module is also configured to determine a bioimpedance waveform based at least partially upon the second signal. The control module is also configured to determine a photoplethysmography (PPG) waveform based at least partially upon the third signal. The control module is also configured to determine a blood oxygen saturation of the patient based at least partially upon the PPG waveform. The control module is also configured to determine a near-infrared spectroscopy (NIRS) waveform based at least partially upon the third signal and the fourth signal. The control module is also configured to determine a tissue oxygen concentration of the patient based at least partially upon the NIRS waveform. The control module is also configured to compare the blood oxygen saturation of the patient to the tissue oxygen concentration of the patient. The control module is also configured to determine a cardiac output of the patient based at least partially upon the comparison.

A method for monitoring a patient is also disclosed. The method includes receiving one or more optical signals from one or more photodiodes. A light emitter is configured to emit light toward a skin of the patient. The one or more photodiodes are configured to measure the one or more optical signals in response to absorption of the light underneath the skin of the patient. The method also includes determining a photoplethysmography (PPG) waveform and a near-infrared spectroscopy (NIRS) waveform based at least partially upon the one or more optical signals. The method also includes determining an arterial blood oxygen saturation based at least partially upon the PPG waveform. The method also includes determining a total blood volume based at least partially upon the NIRS waveform. The method also includes determining a venous blood oxygen saturation based at least partially upon the total blood volume. The method also includes determining an arteriovenous oxygen difference based at least partially upon the arterial blood oxygen saturation and the venous blood oxygen saturation. The method also includes determining a cardiac output of the patient based at least partially upon the arteriovenous oxygen difference.

A method for predicting a cardiac index (CI) for a patient is disclosed. The method includes receiving a set of a measurements from the patient that characterizes an anatomical condition, a physical condition, a physiological condition, or a combination thereof. The method also includes receiving a non-invasive physiological measurement from the patient. The method also includes performing feature extraction on the non-invasive physiological measurement to produce one or more extracted features. The method also includes combining the set of measurements and the extracted features to produce a dataframe. The method also includes predicting the CI for the patient. The CI is predicted using a prediction model based upon the dataframe.

In another embodiment, a method for predicting cardiac indexes (CIs) for a plurality of patients based upon non-invasive physiological measurements from the patients is disclosed. The method includes receiving a first set of measurements from the plurality of patients. The first set of measurements includes invasive physiological measurements of CI. The method also includes receiving a second set of measurements from the plurality of patients. The second set of measurements characterize anatomical conditions, physical conditions, physiological conditions, or a combination thereof. The method also includes receiving a third set of measurements from the plurality of patients. The third set of measurements includes non-invasive physiological measurements. The method also includes performing feature extraction on the third set of measurements to produce extracted features. The method also includes combining the second set of measurements and the extracted features to produce a dataframe. The method also includes training a prediction model based at least partially upon the dataframe to produce a trained prediction model. The method also includes tuning the trained prediction model to produce a tuned prediction model. The method also includes predicting the CIs for at least a portion of the patients using the tuned prediction model based at least partially upon the dataframe.

In another embodiment, the method includes receiving a first set of measurements from the plurality of patients. The first set of measurements includes invasive physiological measurements of CI. The method also includes receiving a second set of measurements from the plurality of patients. The second set of measurements characterize anatomical conditions, physical conditions, and physiological conditions. The anatomical conditions include a presence and a type of congenital heart malformation, a number of functional cardiac ventricles, whether any heart malformation has been repaired, a presence of residual lesions or hemodynamic abnormalities, or a combination thereof. The physical conditions include age, gender, weight, or a combination thereof. The physiological conditions include red blood cell count, hematocrit, or both. The method also includes receiving a third set of measurements from the plurality of patients. The third set of measurements includes non-invasive physiological measurements. The third set of measurements includes non-invasive intra-operative measurements and anesthesia vital measurements. The third set of measurements includes pulse, systolic blood pressure, diastolic blood pressure, heart rate, temperature, respiratory rate, or a combination thereof. The method also includes performing feature extraction on the third set of measurements to produce extracted features. Performing feature extraction includes gating the third set of measurements for a predetermined amount of time to create a time-series, and extracting features from the time-series to produce the extracted features. The extracted features include descriptive summary statistics, autocorrelation, time series quantiles, spectral, Fourier, linear, non-linear, polynomial, wavelet, entropy, or a combination thereof. The method also includes combining the second set of measurements and the extracted features to produce a dataframe. The method also includes predictively imputing data elements that are missing from the dataframe to produce an imputed dataframe. The method also includes combining the first set of measurements and the imputed dataframe to produce a combined dataframe. The method also includes dividing the combined dataframe into a first portion and a second portion. The method also includes performing dimensionality reduction on the first portion of the combined dataframe to produce a reduced dataframe. Performing the dimensionality reduction removes a subset of the data elements from the first portion of the combined dataframe to produce the reduced dataframe. The dimensionality reduction is performed using a random forest tree regressor algorithm. The method also includes training a prediction model based upon the reduced dataframe to produce a trained prediction model. The prediction model is trained using an XGBoost tree algorithm. The method also includes tuning the trained prediction model to produce a tuned prediction model. Tuning the trained prediction model optimizes hyperparameters using a Bayesian search technique by applying a 5-fold cross-validation. The method also includes predicting the CIs for the patients corresponding to the second portion of the combined dataframe to produce predicted Cis. The CIs are predicted using the tuned prediction model.

These and other advantages may be apparent from the following detailed description, and from the claims.

### BRIEF DESCRIPTION OF THE DRAWINGS

FIG. 1A illustrates a graph of a time-dependent waveform of a 6-month, 6-kg infant generated by an ECG sensor, according to an embodiment.

FIG. 1B illustrates a graph of a time-dependent waveform of a 6-month, 6-kg infant generated by a bioimpedance sensor, according to an embodiment.

FIG. 2 includes a graph of a time-dependent waveform of a 6-month, 6-kg infant generated by a functional NIRS sensor, according to an embodiment.

FIG. 3A illustrates a schematic view of a front side (e.g., facing towards the body) of a sensor device (e.g., a wearable sensor insert) worn within a diaper or onesie, where the optical signal is measured from the genitalia, according to an embodiment.

FIG. 3B illustrates a schematic view of a back side (e.g., facing away from the body) of the sensor device (e.g., the wearable sensor insert) worn inside a diaper or onesie, where the optical signal is measured from the genitalia, according to an embodiment.

FIG. 3C illustrates a schematic view of an inside of a casing of the sensor device (e.g., the wearable sensor insert) worn inside a diaper or onesie, according to an embodiment.

FIG. 3D illustrates a schematic view of the front side of the sensor device (e.g., the wearable sensor insert) worn within a diaper or onesie, wherein the optical signal is measured from the genitalia, according to an embodiment.

FIG. 3E illustrates a schematic view of the back side of the sensor device (e.g., the wearable sensor insert) worn within a diaper or onesie, wherein the optical signal is measured from the genitalia, according to an embodiment.

FIG. 4A illustrates a three-dimensional schematic view of a disposable electronic diaper, according to an embodiment.

FIG. 4B illustrates a schematic view of a back view of a reusable case clipped to the electronic diaper, according to an embodiment.

FIG. 4C illustrates a front view of a reusable case clipped to the electronic diaper, according to an embodiment.

FIG. 4D illustrates a bottom view of a reusable case clipped to the electronic diaper, according to an embodiment.

FIG. 4E illustrates a lateral view of a reusable case clipped to the electronic diaper, according to an embodiment.

FIG. 5A illustrates a schematic view of a front side of the sensor device (e.g., a waistband sensor) worn by an infant, according to an embodiment.

FIG. 5B illustrates a schematic view of a back side of the sensor device (e.g., a waistband sensor) worn by an infant, according to an embodiment.

FIG. 6A illustrates a schematic view of an infant wearing the sensor device (e.g., a wearable sensor patch) on the belly, according to an embodiment.

FIG. 6B illustrates a schematic view of an infant wearing the sensor device (e.g., the wearable sensor patch) on the chest, according to an embodiment.

FIG. 6C illustrates a schematic view of an infant wearing the sensor device (e.g., the wearable sensor patch) on the upper back, according to an embodiment.

FIG. 7 illustrates a schematic view of an infant wearing the sensor device (e.g., modular wearable sensor patches), according to an embodiment.

FIG. 8 illustrates a schematic view of a wireless, connected monitoring system that integrates to the patient monitoring system, according to an embodiment.

FIG. 9 illustrates a schematic view of a smart monitoring onesie, according to an embodiment.

FIG. 10 illustrates a graph of the absorption spectra of water, melanin, oxyhemoglobin (HbO2), and deoxyhemoglobin (Hb), according to an embodiment.

FIG. 11 illustrates a schematic process diagram of a patient monitoring system, according to an embodiment.

FIG. 12A illustrates a schematic view of an infant wearing the sensor device (e.g., wearable sensor bands) on both wrists, according to an embodiment.

FIG. 12B illustrates a schematic view of an infant wearing wearable the sensor device (e.g., sensor bands on a wrist and an ankle), according to an embodiment.

FIG. 13 illustrates a table listing monitoring methods in the system with associated physiological signals, according to an embodiment.

FIG. 14A illustrates a schematic view of a PPG and NIRS photodetector configuration including two (e.g., concentric) rings of photodetectors surrounding a central emitter, according to an embodiment.

FIG. 14B illustrates a schematic view of another PPG and NIRS photodetector configuration including an array of photodetectors spiraling outward from the central emitter, according to an embodiment.

FIG. 15 illustrates a flowchart of a method for collecting and/or processing signals using the system, according to an embodiment.

FIG. 16 illustrates a chart showing light attenuation over time for arterial blood, venous blood, and other tissue components, according to an embodiment.

FIG. 17A illustrates a schematic view of the patient monitoring system including two biosensors (e.g., patches including biosensor(s)) attached to the chest and abdomen of the patient, according to an embodiment.

FIG. 17B illustrates a schematic view of the patient monitoring system including a biosensor (e.g., a patch including biosensor(s)) attached to the abdomen of the patient and a pulse oximeter sensor attached to the finger of the patient, according to an embodiment.

FIG. 18A illustrates a schematic view of three light emitting diodes (LEDs) of 650 nm, 800 nm, and 950 nm wavelengths, according to an embodiment.

FIG. 18B illustrates another schematic view of the three LEDs, according to an embodiment.

FIG. 18C illustrates another schematic view of the three LEDs, according to an embodiment.

FIG. 18D illustrates another schematic view of the three LEDs, according to an embodiment.

FIG. 19 illustrates a graph showing the feature importance analysis from an XGBoost model that reveals the significance of 12 variables in influencing the model's cardiac index predictions, according to an embodiment.

FIG. 20 illustrates a graph (e.g., scatter plot) showing a visual comparison between observed and predicted cardiac index (CI) values in the testing cohort, according to an embodiment.

FIG. 21 illustrates a flowchart of a method for predicting CI from non-invasive vital signs, according to an embodiment.

### DETAILED DESCRIPTION

The present disclosure provides a patient health monitoring system that includes a monitoring device that may be worn by a patient. The patient may be an infant or an adult (or anywhere in between). The system and/or device may collect and analyze information from the patient. The information may include images, sounds, numerical data and/or physiological waveforms describing HR, RR, PPG, regional oxygen saturation, temperature, fluid status, motion-related events (e.g., posture), urination, defecation, or a combination thereof. In an embodiment, the system may include three components.

First, the system may include a shell that attaches to the patient and has a form factor such as a patch, band, or garment (e.g., diaper, onesie, sock, etc.). The shell includes at least two conductive electrodes in contact with the patient (e.g., on the chest and/or the abdomen). The shell may be reusable, and the electrodes may be disposable (e.g., wet electrodes made of conductive hydrogel) or reusable (e.g., dry electrodes made of rubber or fabric). The electrodes connect to a control module (e.g., battery powered) located within the shell. The control module may be operated by a programmable microcontroller and features a small-scale, low-power ECG circuit that processes electrical signals collected by the electrodes to determine an ECG waveform. The control module also features a small-scale bioimpedance (BioZ) circuit that injects a low-amperage alternate current of at least one high frequency, and processes the signals collected by the electrodes to determine a BioZ waveform. The alternating electrical current may be from about 1000 Hz to about 100,000 Hz. For example, the electrical current may be from about 1000 Hz to about 10,000 Hz, about 10,000 Hz to about 50,000 Hz, or about 50,000 Hz to about 100,000 Hz. The shell may include an optical module with at least one light emitter (e.g., light emitting diodes) of two wavelengths within the visible-infrared spectrum, and at least two photodetectors. The optical module connects to the control module. The control module may include a circuit that processes the optical signals collected by a photodetector to determine a PPG waveform, and a circuit that processes the optical signals collected by the two photodetectors to determine a NIRS waveform. A three-axis accelerometer within the control module simultaneously measures signals related to the patient's motion (e.g., crawling, shaking), posture (e.g., standing, lying down), and breathing-induced movement of the patient's belly. The microcontroller within the control module collects digital representations of these signals, and then ports them through a wireless interface for further analysis, as described below.

The second component may include a bedside monitoring module (e.g., connected to an infant's crib or hospital bed). For adult patients, the monitoring module can attach, for example, to a nightstand or bed. The monitoring module features a single-board computer and wireless system that collect data transmitted by the control module within the shell described in the paragraph above. Both waveform and numerical data may be sent in a packetized form that is decoded using software operating on the single-board computer. A web/cloud server software program is also coded within this platform and analyzes and then avails information received by the monitoring module to other computing platforms (e.g., computer, cellular phone) connected to the Internet. Such computing platforms may receive information served by the web/cloud server through a wireless interface. The single-board computer also operates algorithms that process signals measured by the various sensors within the electronic diaper to determine or estimate parameters such as HR, HR variability, BP, RR, SV, CO, SpO2, regSpO2, posture, or a combination thereof. The monitoring module also includes an embedded camera (e.g., a web camera) and microphone that collect images and sounds from the infant, and then uses the web/cloud server to avail this information to external Internet-connected devices. The single-board computer also operates algorithms that process the images and sounds of the patient to determine parameters such as activity.

The third component may be or include a “downloadable” software application that operates on a variety of Internet-connected computing platforms to receive and display information from the web/cloud server. Parents of the infant, for example, can download the software application from a website, e.g., one associated with a company providing the above-mentioned components, or a website (e.g., Apple Store, Play Store) that provides multiple software applications that operate on specific devices (e.g., the iPhone or iPad). The software application may feature a graphical user interface (GUI) that renders information collected by the web/cloud server, e.g., images and sounds from the patient, vital signs, motion-related information, and plots of time-dependent waveforms (e.g., ECG waveforms, PPG waveforms) indicating the patient's real-time physiological status. The software application can also process historical patient data. For example, the application can perform a time series analysis of waveforms to obtain statistical parameters (e.g., HR and RR variability), waveform fiducials (e.g., P-Q interval, Q-T interval, QRS complex, pulse transit time), trend data (e.g., evolution of regSpO2), ratios (e.g., EF, T/QRS, regSpO2/SpO2), or output-layer features generated by neural networks (e.g., recurrent neural networks) and network architectures. The software application contains predictive models that can process these parameters alongside the monitored signals to help avert deterioration in the patient's health status. These models, for example, can be used to detect cardiac and hemodynamic irregularities (e.g., arrythmias, sleep apnea) and for the calculation of a “Risk Index” associated to the patient's health. In addition, the software application may include an alarm module that processes one or more of the above-mentioned parameters to generate an audio/visual alarm in the event that the patient is in distress. For example, the alarm module can generate an alarm if the patient's HR, RR, or SpO2 values exceeded a pre-determined threshold. The alarm module can also process a collection of parameters, or trends in these parameters, to determine and predict a relatively complex and dangerous physiological state. In particular, the alarm module includes an algorithm for monitoring trends in HR and RR to predict the onset of sudden infant death syndrome (SIDS), which occurs in about 1 out of every 2000 infants. It may contain an algorithm for monitoring trends in oxygen saturation (SpO2, regSpO2) to identify infant methemoglobinemia (blue baby syndrome), and an algorithm for monitoring CO and tissue perfusion (regSpO2) to predict heart failure in patients with congenital heart disease (CHD), which affects 1 out of every 110 infants.

The software application can render both real-time and historical information which may be viewed by either the patient's parents, or someone associated with them in another capacity, e.g., another family member or pediatrician. The system may include a website that features separate interfaces (e.g., a “family” interface and a “clinician” interface) that are accessed using a specific username/password. Such a system allows remote family members to view the patient, and also facilitates a virtual check-up where a clinician can monitor the patient's cardio-pulmonary behavior by viewing time-dependent waveforms and trends in parameters like HR and RR. The system may also facilitate an artificial check-up where an artificial intelligence algorithm can monitor the patient's cardio-pulmonary health status by analyzing time-dependent waveforms and trends in monitored parameters (HR, RR, SpO2) and digital signal features (as described above). Additionally, because the system includes motion sensors, vital signs and their associated waveforms can be monitored when the patient is relatively motion-free, thus increasing the likelihood that the measured physiological data is not corrupted by motion.

The system may have distinct advantage that it additionally measures real-time physiological information. For example, the system can be installed so that parents can view images, sounds, and vital signs from the infant using their existing smartphone, tablet computer, or laptop computer. These devices can be located at the parent's bedside so that the infant can be monitored during normal sleeping hours. In the unlikely event that a life-threatening physiological event occurs, the software application's alarm module can sound an alarm, allowing the parents or medical clinician to take appropriate action. In another application, the monitoring module may accompany the infant to a day-care facility, allowing the parent to view their child while at work. Conversely, in another application, the monitoring module may accompany the infant from a healthcare facility into the home, allowing the patient's physician to view the patient's health status remotely. In yet another application, a remote family member or local nurse can monitor an aging relative located in an assisted-living facility. In general, using the system described herein, both infant and adult patients can be monitored with a variety of off-the-shelf computing devices from virtually any location having access to the Internet.

More specifically, in one aspect, the system for monitoring a patient may include a garment configured to attach to the patient, and a control module connected to the garment featuring: i) a first sensor that measures heart rate (HR), blood pressure (BP), or parameters used to determine these properties; ii) a second sensor that measures stroke volume (SV), respiratory rate (RR), or a parameter used to determine these properties; iii) a third sensor that measures blood oxygen content (SpO2), tissue oxygen content (regSpO2); and iv) a wireless transmitter configured to receive and wirelessly transmit information from the first, second, and third sensors. The control module interfaces to a monitoring module that is configured to receive information from the first, second, and third sensors through the wireless transmitter, which includes: i) a processing component that processes information generated by the first, second, and third sensors; and ii) a computing component configured to avail content determined by the processing component on a network. A software application operating on a remote computer connects to the network and receives and then displays content availed by the computing component, or parameters calculated therefrom.

The garment may be a onesie configured to be worn over the patient's torso, featuring an outer component configured to attach to the onesie and an inner component configured to contact the patient's skin. The inner component of the onesie may include at least two conductive electrodes, each made from a conductive material, and at least one light source and photodetector. The electrodes, light source, and photodetector may attach to the outer component and be configured to contact the patient's skin.

The first sensor can feature an ECG sensor that connects to the conductive electrodes to receive electrical signals, and then processes these signals with a collection of differential amplifiers and analog filters to generate an ECG waveform.

In another embodiment, the second sensor may include a BioZ sensor that connects to the conductive electrodes to drive and sense electrical signals, and then processes these signals with a collection of differential amplifiers and analog filters to generate a BioZ waveform.

In another embodiment, the third sensor may include an optical sensor that includes a photodiode and a light source (e.g., a light-emitting diode, or LED) in the visible/infrared spectrum. Here, the optical sensor can measure a photoplethysmogram (PPG) from the patient, which is a time-dependent waveform indicating blood flow in an artery or capillary located close to the surface of the infant's skin. Optical wavelengths in the 500-1000 nm range can be used to measure deoxyhemoglobin (Hb), oxyhemoglobin (HbO2), and/or melanin. Melanin can be used to correct the absorption signals for patients of different skin tones.

Algorithms process some or all of the ECG, BioZ, and PPG waveforms using techniques described in detail below to determine HR. Additionally, algorithms process some or all of the ECG, PPG, and BioZ waveforms to determine CO and BP. Additionally, a low-frequency envelope indicating RR is often mapped onto one or both of the ECG, BioZ and PPG waveforms. This envelope can thus be monitored with standard signal processing techniques to determine RR, as is described in more detail below. PPG waveforms measured with both red and infrared LEDs can also be analyzed to determine the infant's value of SpO2 using known techniques in the art.

In another embodiment, a fourth sensor within the control module is an accelerometer (e.g., a three-axis accelerometer) that measures a time-dependent waveform indicating the patient's motion. For example, the accelerometer can measure a time-dependent waveform indicating respiratory-induced motion from the torso. Here, the waveform indicates motion measured along an axis of the accelerometer that is approximately normal to the patient's belly (e.g., within +/−30 degrees of a normal vector extending outward from the infant's belly). In another embodiment, the second sensor associated with the control module includes at least one electrode that measures an electrical impedance change from the patient that varies with respiration rate. Such an electrode, for example, is included in an impedance pneumography sensor. This sensor can be included in the same circuit used to measure ECG waveforms.

In another embodiment, a fifth sensor features a thermal sensor that measures a digital temperature signal indicative of surface and core body temperature patient (e.g., thermoelectric heat flux sensors). Algorithms can process the temperature signals to determine if the patient for conditions such as fever or hypothermia, and determine the estimated volume of blood in the skin.

In another embodiment, a sixth sensor features a thermal sensor that measures a digital temperature signal indicative of urine and/or feces from the patient. The sixth sensor can also include a moisture sensor or a BioZ sensor that measures this parameter. Sensors that monitor surface and core temperature and moisture are embedded in a lower portion of the reusable shell, just underneath the disposable insert, and detect signals related to urine and feces.

The system may have redundancy built into the featured sensors. Sensor redundancy increases signal-to-noise ratio (e.g., multiple photodetectors to detect a stronger PPG waveform) and reduces system failure due to an ill-performing sensor, e.g., one that is damaged, misplaced, uncalibrated.

The monitoring module multiplexes a sensor, in particular the bio-electric and bio-optical sensors and their respective signal activators (e.g., drive electrode current in BioZ, and light emitters in PPG). Multiplexing allows the sensor module to collect signals from multiple locations and from different excitation wavelengths/impedance frequencies avoiding cross contamination. For example, a NIRS signal corresponding to regSpO2 can obtained from PPG waveforms measured at two optode locations (e.g., 1 cm and 5 cm away from the emitter) and two frequencies (e.g., 690 nm and 850 nm). The monitoring module can perform a frequency sweep to obtain spectroscopic measurements of BioZ and PPG signals.

The third sensor may obtain a reflectance and/or transmittance PPG waveform and a NIRS waveform. The PPG waveform may be processed in the monitoring module to calculate blood oxygen saturation (SpO2), a vital sign used as a surrogate for arterial blood oxygenation, i.e., how much oxygen your arterial blood carries. These values can be interpreted by a physician to detect, monitor, and diagnose cardiorespiratory diseases (e.g., chronic obstructive pulmonary disease). The NIRS waveform may be used to extract tissue oxygen concentration (regSpO2), i.e., how much oxygen is reaching the tissue. These values can be interpreted by a physician to detect, monitor, and diagnose problems with oxygen perfusion to tissue (e.g., oxygen delivery to the brain during open heart surgery). SpO2 does not directly correlate with regSpO2, and both signals contain different physiological information about the patient. An algorithm in the monitoring module or the software application compares the disparities in SpO2 and regSpO2 signals. This relationship can be used as indicator of the patient's state of health, for example, a case where blood is being oxygenated but the tissue is not receiving this oxygen may relate to tissue oxygen exchange (e.g., due to low blood pressure, tissue inflammation, low red blood cell count, or low concentrations of hemoglobin in blood). Conversely, when the tissue is receiving as much oxygen as the blood is carrying may relate to blood oxygen content (e.g., venous and arterial blood mixing due to patent ductus arteriosus, single ventricle shunting following surgical repair, reduced blood flow to the lungs, or problems with lung function).

The transmitters that connect the control module to the monitoring module may operate on a wireless protocol based on 802.11 (e.g., WiFi) or 802.15.4 (e.g., Bluetooth®). For example, the wireless transmitter can be a Bluetooth® low-energy transmitter (BLE®), which is optimized to improve battery lifetime.

The processing component within the monitoring module may be a computer (e.g., a single-board computer) that operates a collection of algorithms and software programs. For example, to determine HR, the computer can operate a beat-picking algorithm that analyzes ECG waveforms from the first sensor. Such an algorithm can be the Pan-Tompkins algorithm, or a derivative thereof. As an additional example, the computer can operate an algorithm that analyzes ECG waveforms from the first sensor to determine BP.

In a related embodiment, another algorithm operating in the monitoring module may be a breath-picking algorithm that analyzes waveforms modulated by the patient's breathing patterns to determine RR. For example, the breath-picking algorithm can operate a slope-summing function, or a derivative thereof. The slope-summing algorithm may be applied to a continuous blood pressure waveform to determine heartbeat-induced pulses, but the same methodology can also be applied to waveforms modulated by breathing patterns to measure RR.

In a related embodiment, another two algorithms operating in the monitoring module are a beat-picking algorithm and a breath-picking algorithm that analyzes BioZ waveforms from the second sensor to determine HR and RR.

In a related embodiment, another algorithm operating in the monitoring module analyzes the PPG waveform to determine the concentration of certain molecules (e.g., Hb, HbO2, H2O) therein. The concentration of these molecules may be calculated according to the Beer-Lambert Law, where: Absorbance (A)=Molar Attenuation Coefficient (ε)*Optical Path Length (L)*Concentration of Molecule (c), or a derivative thereof, to determine oxygen concentration in blood (SpO2) and tissue (regSpO2). Additional algorithms may include a beat-picking algorithm and a breath-picking algorithm that analyzes PPG waveforms from the third sensor to determine HR and RR.

In a related embodiment, another algorithm operating in the monitoring module analyzes the interplay between the optical (PPG) and electrical (ECG, BioZ) signals. This algorithm can more accurately estimate patient physiological variables such as CO and BP by finding the common elements of one or more of those signals and adjusting for variance due to noise. For example, the pulsatile component of the ECG signal corresponding to cardiac pumping correlates with the pulsatile elements of the PPG and BioZ signals.

This algorithm includes information from other sensors. A thermal sensor can measure temperature (e.g., core body, skin) as a surrogate for metabolic activity and vascular thermoregulation (which causes changes in vascular resistance in the skin and underlying tissue due to vasocontraction and vasodilation), and use this data to improve the estimates of CO and BP. Temperature can also affect NIRS signals obtained from a PPG waveform by changing the absorbance of water (H2O) with temperature.

An accelerometer can monitor patient activity (e.g., posture, motion artifacts, sleeping, eating, physical exercise) and use this data to improve the estimates of CO and BP, as shown in the equation below:

$$F \{ {f}_{1} ( ECG ) , {f}_{2} ( PPG ) , {f}_{3} ( BioZ ) , {f}_{other} ( Other ) \} \to ( Cardiac Output , Blood pressure ) \qquad ( 4 )$$

where F{ . . . } is a function that maps its contents to CO and BP, fᵢ(X) represents a function i that processes a signal X, and “Other” represents signals from other sensors, for example, temperature and motion.

The monitoring module includes a camera (e.g., a web camera that captures real-time video images of the patient), and a microphone that captures voice signals indicating (e.g., that an infant is crying). The web camera integrates directly with the single-board computer within the monitoring module. In this case, the computer also operates a web/cloud server that serves up content which can be viewed with a remote, Internet-connected device. For example, the content can be one of the following: an image, a vital sign, a time-dependent physiological waveform, a motion waveform, a motion-related parameter, a posture, or an indication if the patient is sleeping. In embodiments, the web/cloud server connects to a website, from which content can be viewed through an in-home wireless network connected to the Internet. The content can be viewed by any Internet-connected computing platform using the downloadable software application. Such computing platforms include a desktop computer, laptop computer, tablet computer, cellular telephone, smartphone, or similar device. Such systems may include a high-resolution video camera that yields high-quality color images of the infant that can be viewed from either home or work. During night, when the infant is typically sleeping, the computing platform can be located by a parent's bedside.

The software application may be configured to be downloaded from a website operating on the Internet. It may include a GUI that displays an image and at least one of a vital sign, a time-dependent physiological waveform, a motion waveform, a motion-related parameter, a posture, an indication if the infant is sleeping, and a PP parameter.

The software application includes a section to set and/or select alarm parameters, e.g., those associated with vital sign values and patient activity (e.g., whether or not the patient is sleeping, the patient's posture and motion, and feces/urine discharge), and time-dependent trends and/or combinations of these properties. Another algorithm in the downloadable software application processes the data from the monitoring module and predicts the health status of the patient, e.g., by calculating a “Health Risk” score. Another algorithm in the software synthesizes continuous monitoring data and historical data, e.g., derivatives or integrals of the signals/data over a period of time, and statistics and statistical comparisons between and within the parameters monitored. The information produced by the software application provides a user (e.g., the patient's physician) information to perform an assessment of, for example, cardiac function.

The system can integrate with any Internet-based system, e.g., a website. The website includes a first user interface associated with the patient's family, and a second user interface associated with a medical clinician. The clinician, for example, can be a pediatrician, a general physician, or a nurse or assistant working at an assisted-living facility for adults. The second interface may also be associated with a plurality of patients, allowing the clinician to check up on one patient from a group of patients. This allows, for example, the pediatrician to check the infant's vital signs, waveforms, crawling and/or sleeping behavior, and a variety of other parameters related to the infant's physiology and behavior. For example, during such a procedure, the pediatrician may evaluate trends in the infant's HR and RR values and observe their ECG waveforms to detect cardiac abnormalities. Similarly, algorithms operating with the software application can analyze motion waveforms generated by the accelerometer within the electronic diaper to indicate to the pediatrician if the infant is crawling, sleeping, or moving about in a normal manner.

The system and method described herein provide real-time monitoring of a patient using a combination of video images, sound, vital signs, motion, and PP parameters. Such information can be processed with software associated with hospital-grade vital sign monitors to detect and predict when the patient is in need of medical attention, or simply when a diaper needs to be changed. In one sense, the system and method bring aspects of sophisticated medical care normally conducted in the NICU to the home environment. This can potentially empower family members to provide more sophisticated care for their own infant, while also providing data that a clinician can use to make an effect, remote diagnosis.

There are also advantages associated with the form factor of the electronic diaper. As described herein, it includes a large reusable shell, and a small disposable insert that gets soiled during a PP event. This means only a small part of the diaper gets thrown away after such an event occurs. Ultimately, this helps to reduce the substantial waste associated with disposable diapers. Additionally, the disposable insert can be composed exclusively of biodegradable materials which quickly degrade in landfills. This helps reduce the environmental impact of the disposable insert compared to conventional disposable diapers, which typically include plastic materials which can literally take hundreds of years to degrade.

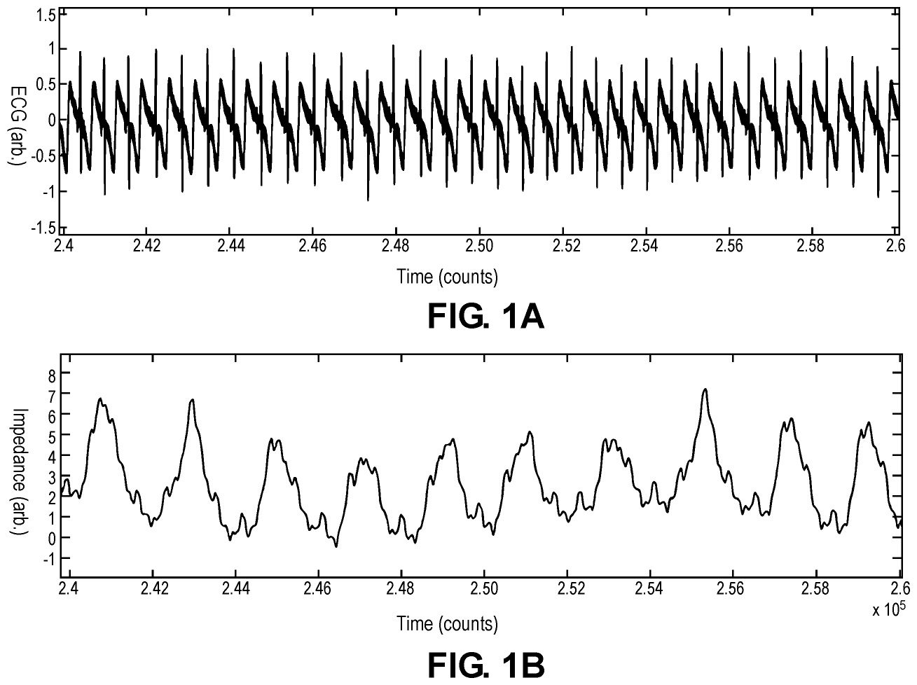

FIGS. 1A and 1B show time-dependent waveform signals obtained from a plurality of electrodes (e.g., four electrodes) attached to a 6-month, 6-kg infant. More particularly, FIG. 1A is a graph of an electrocardiogram (ECG) signal from an ECG sensor in contact with the infant, where its periodicity signal corresponds with the infant's heart rate, which is faster than that of adults. FIG. 1B is a graph of a bioimpedance (i.e., BioZ) signal from a BioZ sensor in contact with the infant, to which the higher amplitude, lower frequency signals correspond. The ECG sensor and/or the BioZ sensor may be coupled to and/or positioned within the sensor device described below.

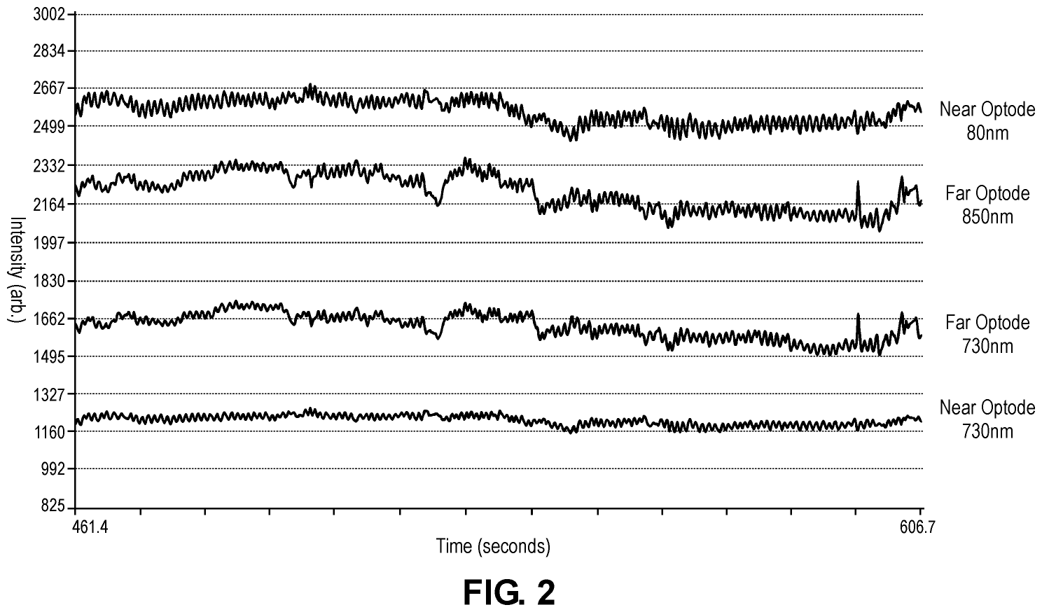

FIG. 2 includes a graph of a time-dependent waveform of the 6-month, 6-kg infant generated by a functional near-infrared spectroscopy (NIRS) sensor. The sensor may be coupled to and/or positioned within the sensor device. The emitter wavelengths are 730 nm and 850 nm. The two detectors correspond to a tissue probing optode (labelled “Far Optode”) and a skin reference optode (labelled “Near Optode”).

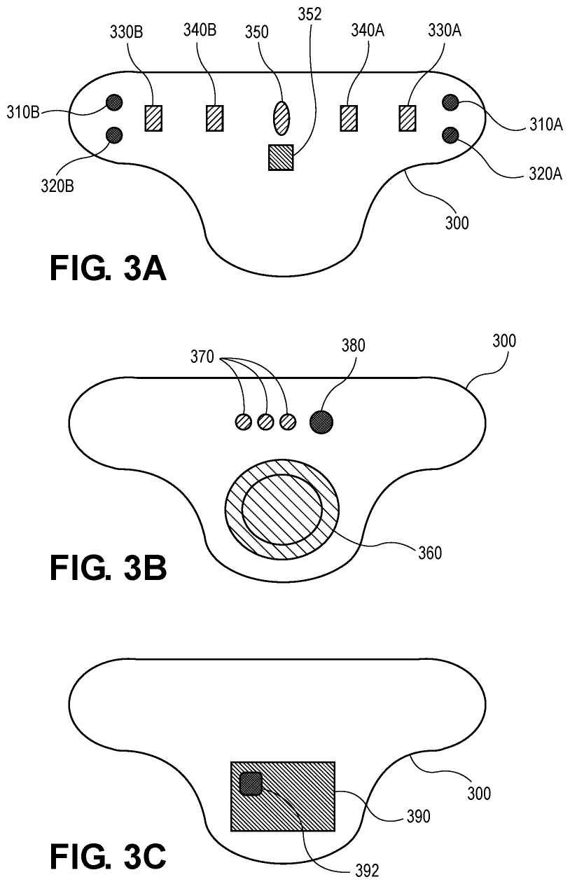

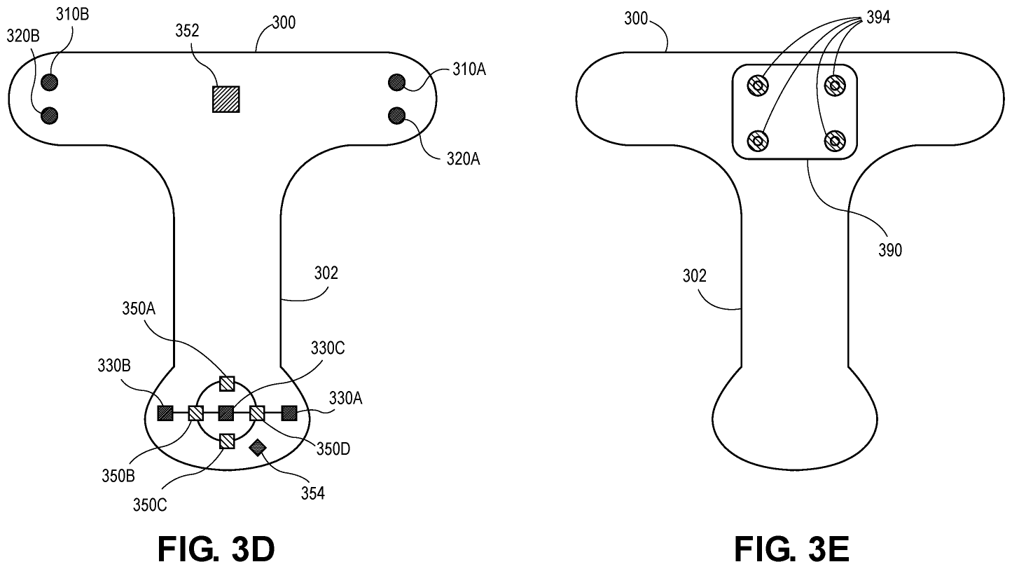

FIGS. 3A-3E illustrate a sensor device 300 that may be worn within an item of clothing (e.g., a diaper or onesie). The sensor device 300 may be or include an insert (e.g., a shell) that may be inserted and/or positioned at least partially within the item of clothing. As shown in FIG. 3A, the front of the sensor device 300 may include one or more sense electrodes (two are shown: 310A, 310B) and one or more drive electrodes (two are shown: 320A, 320B). The sensor device 300 may also include one or more far photodetectors (two are shown: 330A, 330B) and one or more near photodetectors (two are shown: 340A, 340B). The sensor device 300 may also include a red and/or near-infrared LED emitter 350 and a temperature sensor 352.

As shown in FIG. 3B, the back of the sensor device 300 may also include one or more inductive charging rings 360, one or more light-emitting diode (LED) indicators 370, and a button input 380.

As shown in FIG. 3C, the (e.g., inside of the) sensor device 300 may include a printed circuit board 390 including a controller, a battery, and/or wireless communication module. The sensor device 300 may also include an embedded accelerometer 392.

As shown in FIG. 3D, the front of the sensor device 300 may include a flexible connector 302. This embodiment of the sensor device 300 may also include a plurality of red and/or near-infrared LED emitters 350A-350D as well as a moisture sensor 354. The photodetectors 330A-330C may be placed on and/or over the genitalia (e.g., an area of high vasculature).

As shown in FIG. 3E, the back of the sensor device 300 may include the printed circuit board 390 including a microcontroller, accelerometer, wireless communication module, and battery. The sensor device 300 may also include one or more magnets 394 for a charging port attachment. The magnets 394 may also serves as a detachable case.

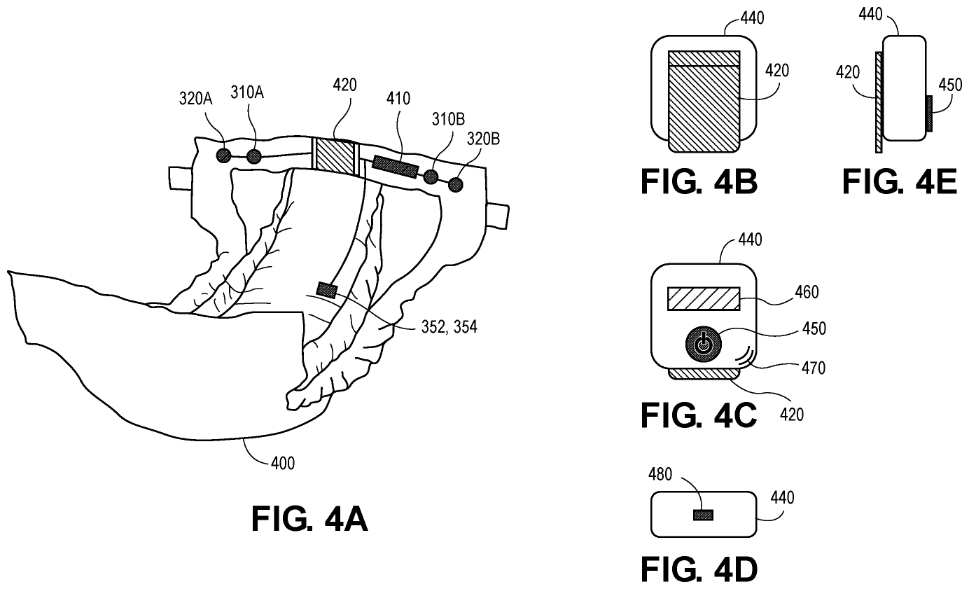

FIGS. 4A-4E illustrate a disposable electronic diaper 400 with at least a portion of the sensor device 300 coupled thereto and/or positioned therein. As shown in FIG. 4A, in addition to the electrodes 310A, 310B, 320A, 320B, the sensor device 300 may also include a NIRS band 410 with an emitter and/or detector(s). The sensor device 300 may also include a clipping area 420. In alternative embodiments, reference number 420 may also or instead include conductive ink, tape, VELCRO®, or the like. This area may allow for the exchange of power and/or information between sensors in the sensor device 300 and the diaper 400.

As shown in FIG. 4B, a reusable casing 440 may be clipped to the diaper 400. The casing 440 may include the accelerometer mentioned above. The casing 440 may also include the clipping area 420 for attachment to the diaper 400. The clipping area 420 may include sensors directed to the body such as temperature sensors, optical sensors, ECG sensors, ultrasound sensors, or the like.

As shown in FIG. 4C, the casing 440 may include a button input 450, a visual display screen 460, and an audio module 470 that may include a microphone input and/or speaker output.

As shown in FIG. 4D, the casing 440 may include a charging port 480. As shown in FIG. 4E, the casing 440 may include the clipping area 420 and the button input 450.

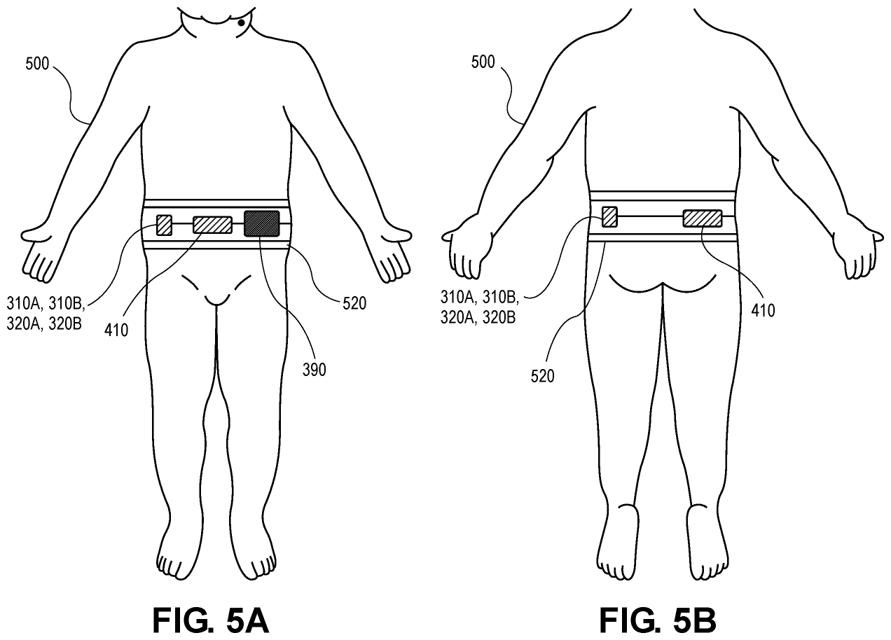

FIGS. 5A and 5B illustrate a front side and a back side of the sensor device 300 worn by a patient (e.g., an infant) 500. The sensor device 300 may be worn on a waistband (e.g., a belt) 520 and thus be referred to as a waistband sensor. The waistband 520 and/or sensor device 300 may include the NIRS sensor 410 and the microcontroller 390.

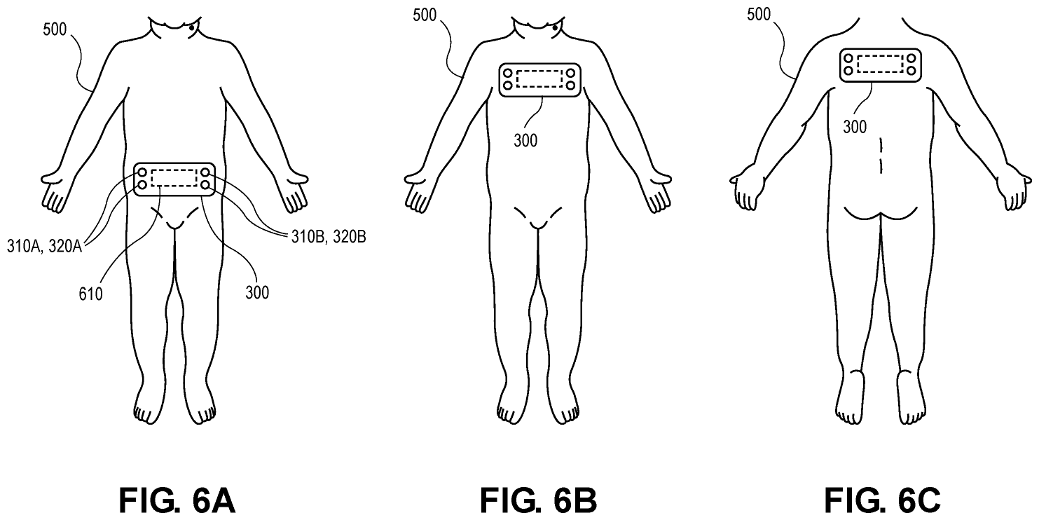

FIGS. 6A-6C illustrate the sensor device 300 being attached to the belly (FIG. 6A), the chest (FIG. 6B), and the back (FIG. 6C) of the patient 500. In the embodiment shown, the sensor device 300 may be or include a patch that may be configured to adhere or otherwise contact the patient 500. The sensor device 300 may include the electrodes 310A, 310B, 320A, 320B, the electronics (e.g., microcontroller and/or communication module) 390, and an optical sensor 610.

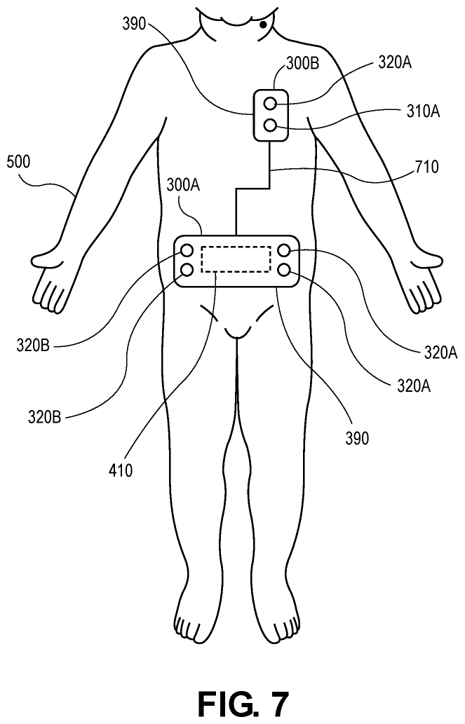

FIG. 7 illustrates the patient 500 wearing one or more sensor device 300A, 300B (e.g., wearable patches on the belly and the chest). The sensor devices 300A, 300B may be connected via one or more wires 710, or they may be configured to communicate with one another wirelessly.

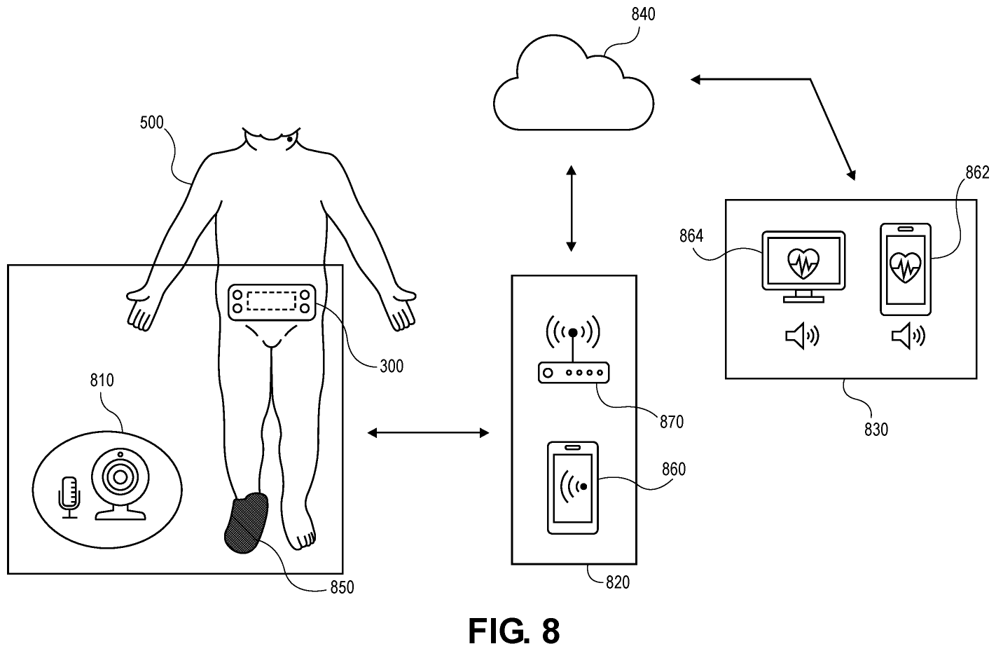

FIG. 8 illustrates a system including the sensor device 300. The system may include a microphone and/or camera 810 for crib-side monitoring. The system may also include a gateway 820 to interface between the sensor device 300 and a cloud data storage 840. The gateway 820 may include a smartphone 860 and/or a WIFI router 870. The system may also include a visual and/or audio display such as a smartphone 862 and/or a computer 864 to access and display data such as graphs, charts, and alarms that can be adapted to a user such as a physician, parents, and/or a patient. The system may also include a smart sock 850 with sensors such as electrodes, optodes, thermometers, and the like that can measure HR, SpO2, and peripheral body temperature.

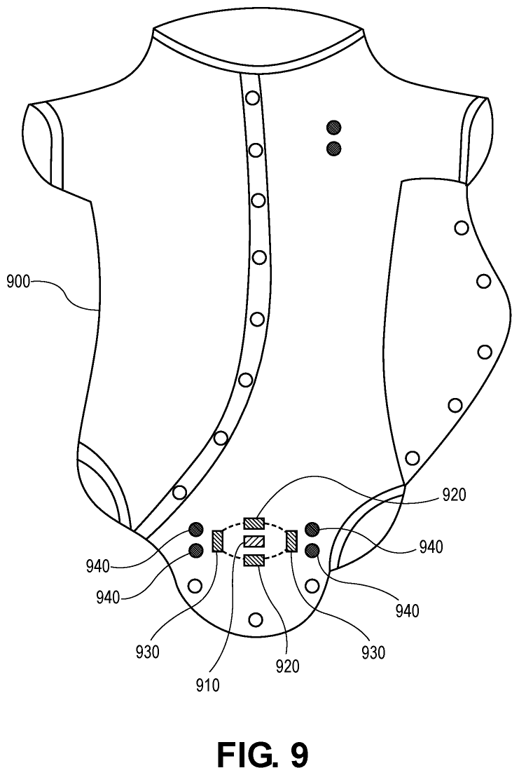

FIG. 9 illustrates a smart monitoring onesie 900. The onesie 900 may include an LED emitter 910, one or more near photodetectors 920, one or more far photodetectors 930, and one or more electrodes 940.

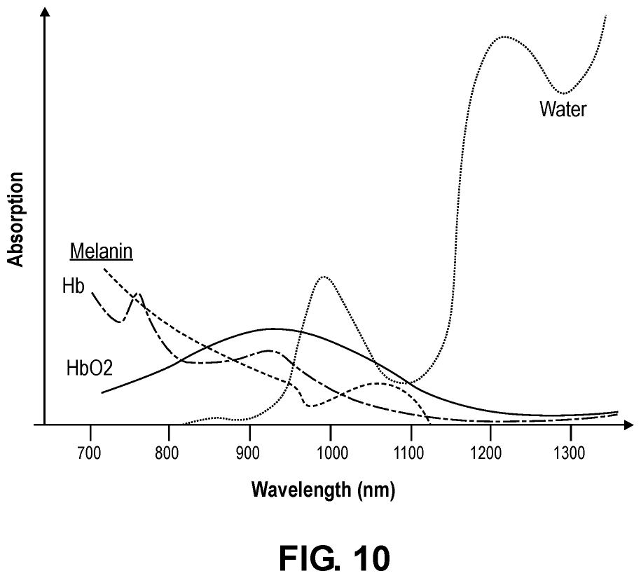

FIG. 10 illustrates a graph of the absorption spectra of water, melanin, oxyhemoglobin (HbO2), and deoxyhemoglobin (Hb).

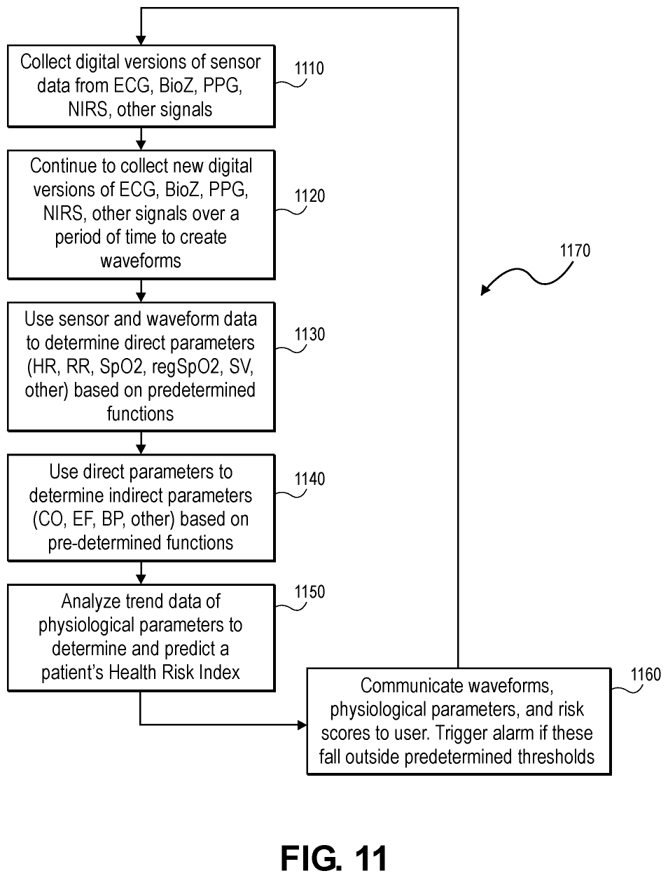

FIG. 11 illustrates a flowchart of a method for monitoring a patient. The method may include collecting digitally-converted data from a sensor module, as at 1110. The method may also include (at further time stamps) continue to collect digitally-converted data from the sensor module for a predetermined period of time to generate waveforms of the monitored parameters, as at 1120. The method may also include using the digital sensor data and waveform data to determine direct physiological parameters (e.g., HR, RR, SpO2, regSpO2, SV, other) based on predetermined functions, as at 1130. In an alternative embodiment, the method may include extracting more complex biomarkers from these waveforms using pre-determined artificial intelligence algorithms. The method may also include using the digital sensor data, waveform data, determined physiological parameters, and/or waveform fiducials to determine indirect physiological parameters (e.g., CO, EF, BP, other) based on pre-determined functions, as at 1140. The method may also include using pre-determined functions to analyze trends of physiological parameters to determine a patient's Health Risk Index based on the patient's current status, as at 1150. For example, this may include determining when the patient is not breathing or the patient's heart has stopped. In another example, this may include analyzing trends of physiological parameters and health risk index to predict a patient's future deterioration (e.g., heart failure, lowering oxygen, increase in temperature). In another example, this may include determining a cardiac output for the patient. The method may also include communicating suitable digital data waveforms, determined physiological parameters, and health risk scores to a user, as at 1160. In one embodiment, this may include triggering an alarm if these variables fall outside predetermined thresholds. The method may also include continuing to collect data, process the data, and display the data, as at 1170.

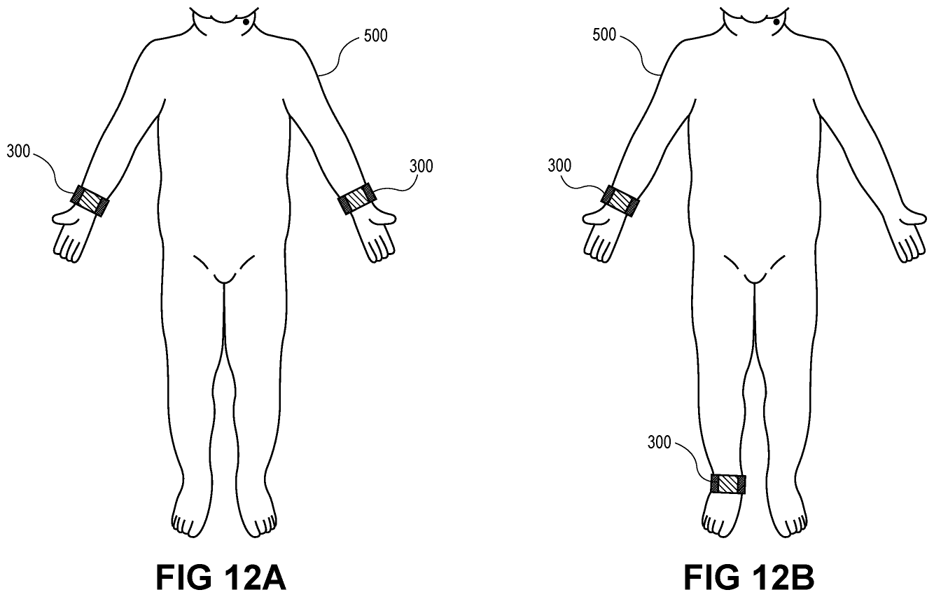

FIGS. 12A and 12B illustrate the patient (e.g., an infant) 500 wearing the sensor device 300 on the wrist and/or ankle. For example, the sensor device 300 may be or include a wristband and/or watch having an elastic band, and a casing including a battery, a microcontroller, a wireless communication module, optical sensors, and electrodes. As shown in FIG. 12B, the sensor device 300 may also or instead be worn on the ankle.

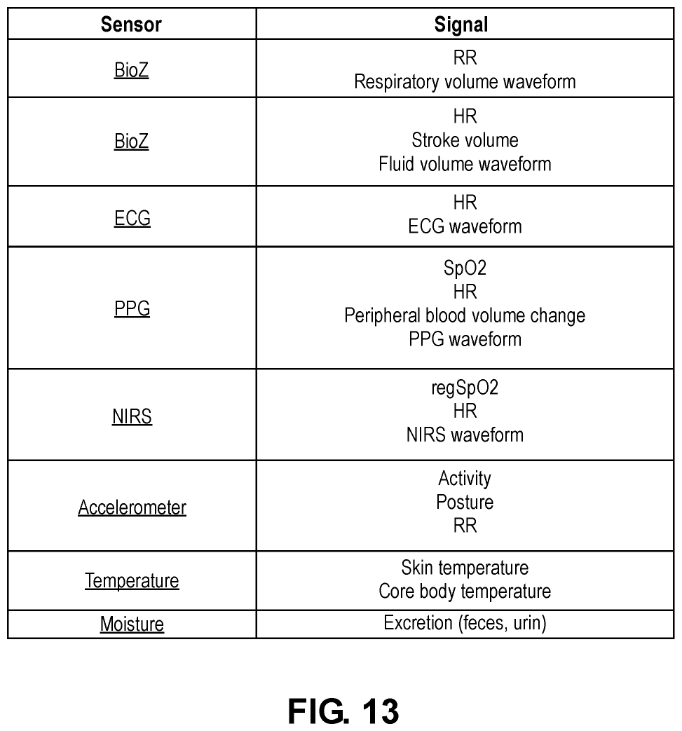

FIG. 13 illustrates a table including a plurality of monitoring methods and their associated physiological signals.

Thus, as mentioned above, a system for monitoring an infant or adult patient may include an attachment to the infant, and a control module connected to the garment featuring: i) a first sensor configured to measure at least one of heart rate or a parameter used to determine heart rate; ii) a second sensor configured to measure bioimpedance; iii) a third sensor configured to measure oxygen saturation in blood or a region of tissue; and iv) a wireless transmitter configured to receive and wirelessly transmit information from the first, second, and third sensors.

The system and method may combine some of the functionality of medical-grade vital sign monitors with that of consumer computing platforms, and bring this solution into the home to monitor infants. Thus, the system and method can include many of the capabilities of monitors which are used in the hospital or with high-end telemedicine systems. For example, the system and method can measure high-quality ECG waveforms, which can then be sent through the Internet to a web-based system that can be viewed by a pediatrician and used to monitor the infant's cardiac performance. In other embodiments, trends in the infant's vital signs can be transported and analyzed in a similar manner to diagnose certain medical conditions. Motion-related properties, such as how often an infant is crawling, or their posture, can also be analyzed in this way to determine if the infant's motor skills are developing in a normal way. The system and method described herein allows an infant to be monitored in the comfort of home in much the same way that the infant can be monitored in the hospital.

The infant-monitoring system can feature a computing platform that connects to the Internet, and thus the features of such systems can be incorporated to help improve infant monitoring. For example, using an accompanying web-based system, the shell and monitoring module can be deployed to monitor an infant in one location (e.g., a daycare center), while the remote computer can be deployed in virtually any other location with Internet connectivity so that the infant can be observed. This allows, for example, the infant to be viewed by family members, medical professionals, and/or research scientists. In another embodiment, the remote computer can be used to download sounds, music, or educational content from the Internet, and then transfer these to the monitoring module for playback.

In other embodiments, the monitoring system can be deployed in a form factor other than that described above. For example, rather than featuring a relatively large reusable shell and a relatively small disposable insert, the electronic garment can include a disposable element similar to diapers available today (e.g., diapers made under the Huggies or Pampers brand) that includes a small, discrete insert for the monitoring module. Here, the disposable diaper may include integrated electrodes (e.g., including materials such as conductive rubber or conductive fabric) that connect to the control module through a simple connector. In this embodiment, the control module may be encased in a durable, waterproof housing that allows it to withstand day-to-day abuse by the infant. In still other embodiments, the control module may be integrated with a reusable cloth garment that may be washed in between uses. The scope extends to any form factor that combines the biosensors described with a control module and algorithms described herein, and then couples the control module to a monitoring module and downloadable software interface as described herein. Other embodiments are within the scope of the following claims.

### Optical Monitoring System of Arteriovenous Oxygen Difference

The system (e.g., including the sensor device 300 and/or diaper 400) may also or instead use the NIRS sensor(s) and/or the PPG sensor(s) to measure an arteriovenous oxygen (A-V O₂) difference in the patient, which may be a surrogate of cardiac output. The system may include filters that can process the optical signals from the NIRS sensors and/or the PPG sensors to discern between the triphasic pulsatile arterial signal, and the monophasic non-pulsatile venous signals. The pulse wave of the signal can also be used to identify retrograde blood flow, for example, in Fontan circulation.

The PPG waveform may also be used to obtain arterial blood oxygen saturation (e.g., SpO2 and/or SaO2). More particularly, the NIRS signal may be used to obtain the total amount of hemoglobin, HbT, and/or the total blood volume. Venous blood oxygen saturation (i.e., SvO2) may be calculated from the arterial blood oxygen saturation, blood oxygen saturation, and/or total blood volume.

The Fick equation may be used to relate the cardiac output, the rate of oxygen consumption, and/or the arteriovenous oxygen content difference. In Fick, the rate of oxygen consumption (VO2) equals cardiac output (CO) times the arteriovenous content difference A-V O₂. VO₂ can be estimated by nomogram, and/or measured using a calorimetry device. CO, which equals stroke volume (SV) times heart rate (HR), can be measured non-invasively with bioimpedance. Knowing two of the variables in the Fick Equation gives an exact measure of the other. Knowing only A-V O₂ can serve as an estimate for cardiac output. The system may be used as a trend monitor, where a baseline signal is obtained for the patient. Furthermore, mixed venous oxygen saturation may drop when CO is low, which may increase the AVO₂.

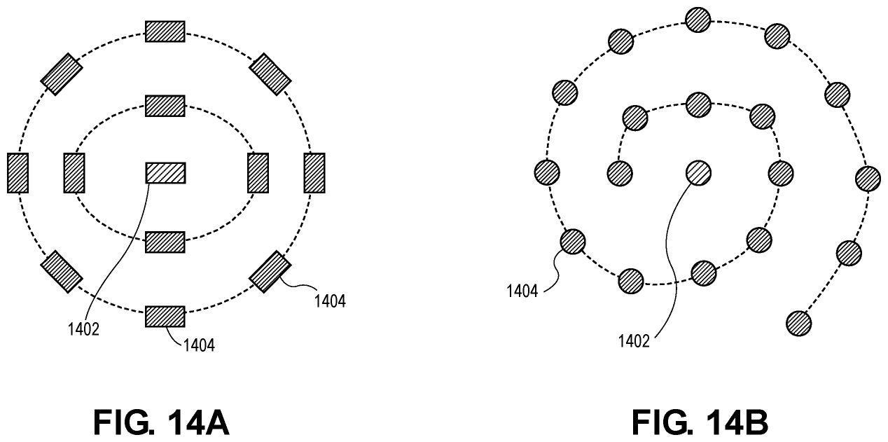

FIG. 14A illustrates a schematic view of a PPG and NIRS photodetector configuration including two (e.g., concentric) rings of photodetectors 1404 surrounding a central emitter 1402, according to an embodiment. The configuration shown in FIG. 14A may be part of (e.g., coupled to) the sensor device 300 and/or the diaper 400, or the configuration may be directly attached to the patient 500 without the sensor device 300 and/or the diaper 400.

The configuration may include one or more emitters 1402. The emitter 1402 may be or include a light emitting diode (LED) that is configured to emit light in the red and/or infrared wavelength. The configuration may also include one or more optodes 1404. The optodes 1404 may be arranged in one or more rings. For example, there may be a first (e.g., inner) ring of optodes 1404 around the emitter 1402, and a second (e.g., outer) ring of optodes 1404 around the inner ring and the emitter 1402. Having the emitter 1402 and optodes 1404 arranged in this manner may provide the spacing for NIRS, allow for spatial information to be detected, and increase the signal-to-noise (SNR), especially in areas of lower vasculature.

FIG. 14B illustrates a schematic view of another PPG and NIRS photodetector configuration including an array of photodetectors spiraling outward from the central emitter, according to an embodiment. The configuration in FIG. 14B may also include the emitter 1402 and the optodes 1404; however, the optodes 1404 may be arranged in a spiral pattern from the central emitter 1402. Having the emitter 1402 and optodes 1404 arranged in this manner may provide the spacing for NIRS, allow for spatial information to be detected, and increase the signal-to-noise (SNR), especially in areas of lower vasculature. In addition, this pattern may also provide higher spatial resolution due to the interpolation of the signal in this pattern.

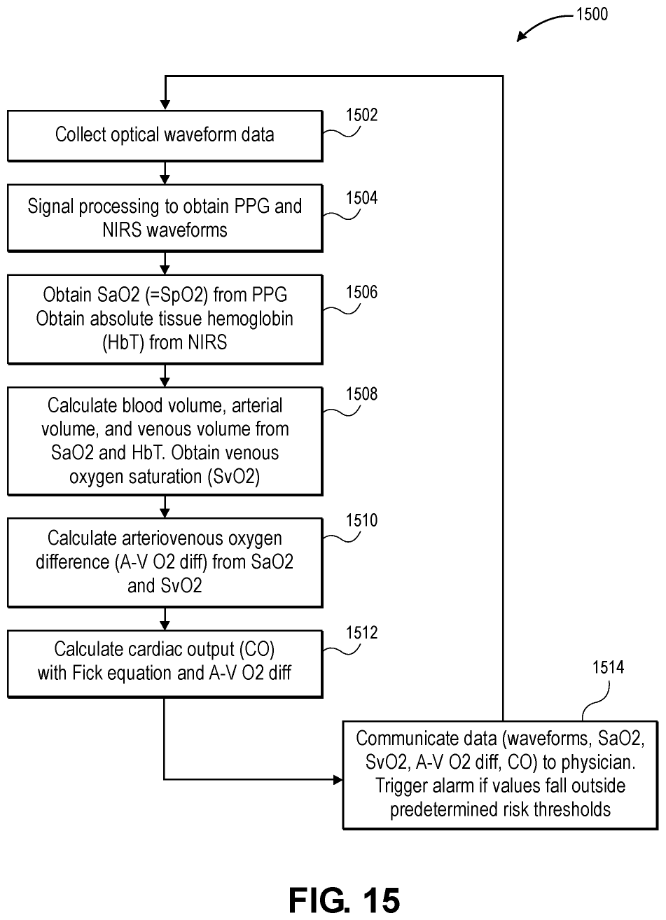

FIG. 15 illustrates a flowchart of a method 1500 for collecting and/or processing signals using the system, according to an embodiment. The method may include measuring raw optical data from the optical sensors (e.g., emitter 1402 and/or optodes 1404), as at 1502.

The method may also include processing the raw optical data with filters to resolve the PPG signal and/or NIRS signal therefrom, as at 1504.

The method may also include determining an SpO₂ oxygen saturation based at least partially upon the PPG waveform, as at 1506. The SpO₂ oxygen saturation may be substantially equivalent to the arterial oxygen saturation (SaO₂). In addition, the total tissue hemoglobin (HbT) may be determined based at least partially upon the NIRS waveform.

The method may also include determining the blood volume, arterial blood volume, and/or venous blood based at least partially upon the SaO₂ and/or the HbT, as at 1508. The blood volume, arterial blood volume, and/or venous blood may also or instead be determined based at least partially upon the bioimpedance and/or temperature of the patient. The venous oxygen saturation (SvO₂) may be determined based at least partially upon the oxygen saturation of the venous blood.

The method may also include determining the A-V O₂ difference by subtracting the SvO₂ from the SaO₂, as at 1510.

The method may also include determining the cardiac output of the patient based at least partially upon the A-V O₂ difference, as at 1512. The cardiac output may also or instead be determined based at least partially upon a nomogram-derived or measured VO₂. Conversely, the VO₂ may be determined when the cardiac output is known (e.g., by using a bioimpedance device to measure stroke volume and/or by thermodilution). Alternatively, the A-V O₂ may be used as a surrogate for the cardiac output, or as a surrogate for metabolic activity. The cardiac output may be determined using the Fick equation.

The method may also include transmitting one or more of the parameters measured and/or determined above to a user (e.g., the patient or the patient's doctor or family), as at 1514. The parameters may be or include waveforms, data trends, etc.

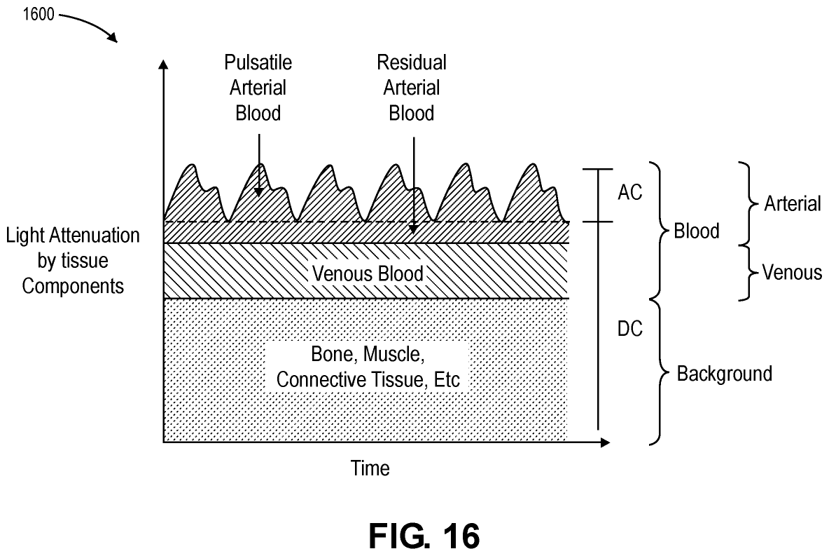

FIG. 16 illustrates a chart 1600 showing light attenuation over time for arterial blood, venous blood, and other tissue components, according to an embodiment.

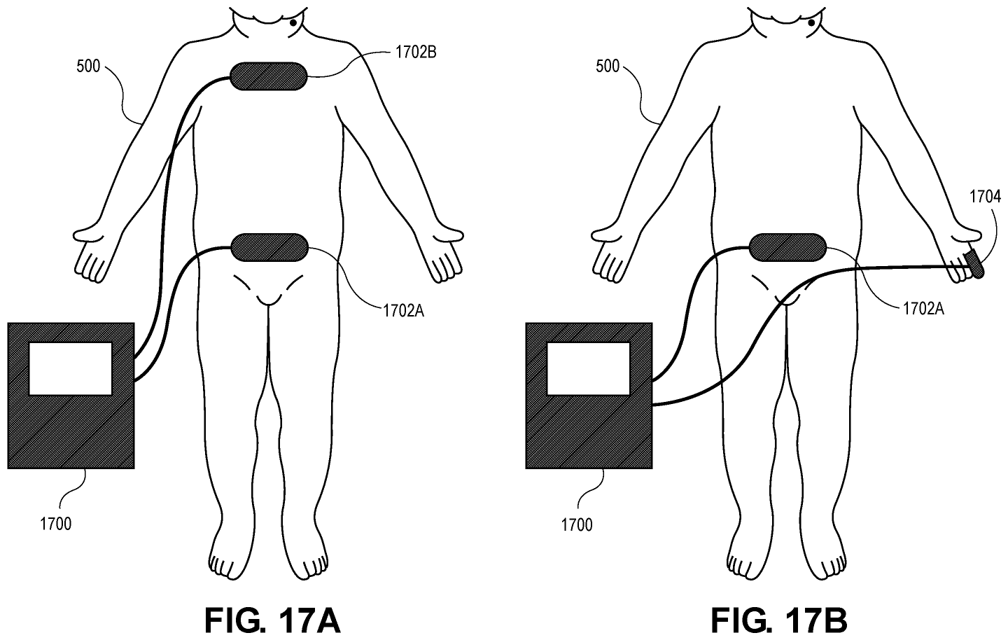

FIG. 17A illustrates a schematic view of the patient monitoring system 1700 including two biosensors (e.g., patches including biosensor(s)) attached to the chest and abdomen of the patient 500, according to an embodiment. The patient monitoring system 1700 may include a processor and a display. The patient monitoring system 1700 may include or be connected to a first optical biosensor patch 1702A that may be attached to the patient's abdomen. The first biosensor 1702A may be configured to measure PPG and/or NIRS signals in the abdominal region. The patient monitoring system 1700 may also include or be connected to a second optical biosensor patch 1702B that may be attached to the patient's chest. The second first biosensor 1702B may be configured to measure PPG and/or NIRS signals in the chest region. The biosensors 1702A, 1702B may include BioZ electrodes to measure the bioimpedance across the tissue portion of the patient under the sensor and/or the BioZ across the tissue portion of the patient.

FIG. 17B illustrates a schematic view of the patient monitoring system 1700 including a biosensor (e.g., a patch including biosensor(s)) attached to the abdomen of the patient and a pulse oximeter sensor attached to the finger of the patient, according to an embodiment. The system 1700 may include or be connected to a pulse oximeter 1704 that may be attached to the patient's index finger. The patient monitoring system 1700 may be configured to provide cardiac, vascular, and/or respiratory monitoring of the patient. In another example, the patient monitoring system 1700 may be configured to provide abdominal monitoring for necrotizing enterocolitis when the patient is a preterm infant. These measurements may provide a physician with information to identify low intestinal perfusion and/or low cardiac output.

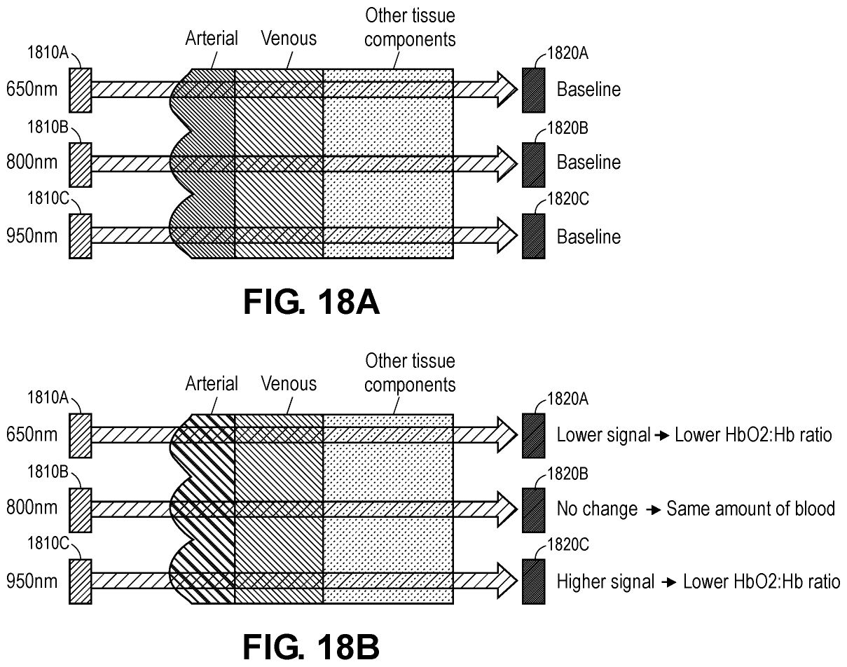

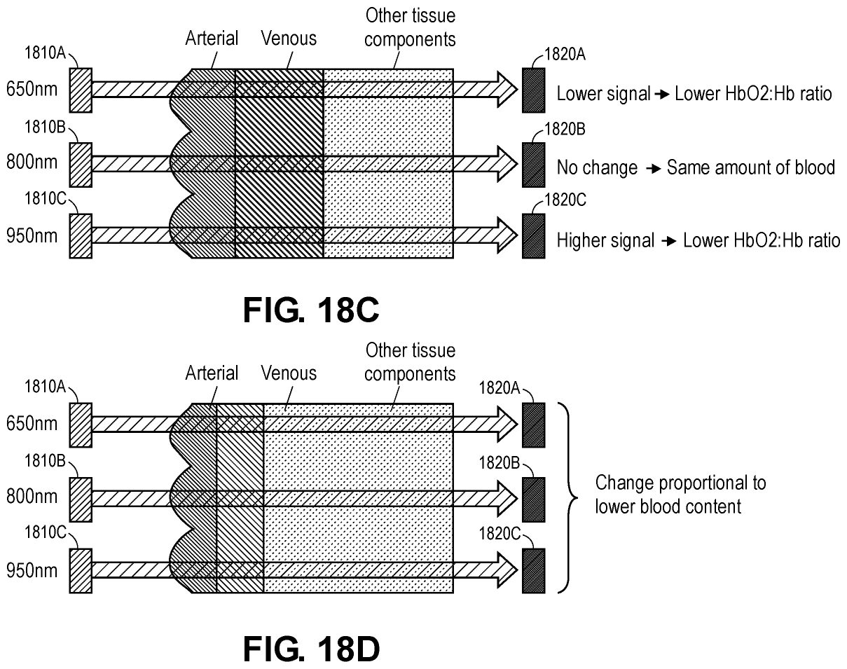

FIG. 18A illustrates a schematic view of three light emitting diodes (LEDs) 1810A-1810C of 650 nm, 800 nm, and 950 nm wavelengths, according to an embodiment. The LEDs 1810A-1810C are emitting light that is attenuated in the patient's tissue, which contains pulsatile arterial blood, non-pulsatile venous blood, and other tissue components. The light is then detected by optodes 1820A-1820C and used to produce a baseline signal.

FIG. 18B illustrates another schematic view of the three LEDs 1810A-1810C, according to an embodiment. The LEDs 1810A-1810C are emitting light that is attenuated in the patient's tissue, which contains pulsatile arterial blood, non-pulsatile venous blood, and other tissue components. The light is then detected by optodes 1820A-1820C and used to produce a signal revealing a low (e.g., arterial) oxygen saturation.

FIG. 18C illustrates another schematic view of the three LEDs 1810A-1810C, according to an embodiment. The LEDs 1810A-1810C are emitting light that is attenuated in the patient's tissue, which contains pulsatile arterial blood, non-pulsatile venous blood, and other tissue components. The light is then detected by optodes 1820A-1820C and used to produce a signal revealing a low (e.g., venous) oxygen saturation.

FIG. 18D illustrates another schematic view of the three LEDs 1810A-1810C, according to an embodiment. The LEDs 1810A-1810C are emitting light that is attenuated in the patient's tissue, which contains pulsatile arterial blood, non-pulsatile venous blood, and other tissue components. The light is then detected by optodes 1820A-1820C and used to produce a signal revealing a low blood content in the tissue.

In an embodiment, the system may be used to monitor a patient and may include a NIRS sensor and a PPG sensor that are each configured to measure optical signals from the patient. The system may filter and/or process the signals to obtain one or more single data points or waveforms from one or more of: arterial oxygen saturation, total hemoglobin content, total blood content, blood oxygen saturation, venous oxygen saturation, or a combination thereof. The data (e.g., data points and/or waveforms) may be used to measure/estimate the A-V (arterial-venous) ratio of the patient. The data (e.g., data points and/or waveforms) may be used to measure/estimate the residual arterial blood volume of the patient. The data (e.g., data points and/or waveforms) may be used to measure/estimate the peripheral arterial tonometry of the patient. The data (e.g., data points and/or waveforms) may be used to measure/estimate the arteriovenous oxygen difference (A-V O₂) of the patient. The data (e.g., data points and/or waveforms) may be used to measure/estimate the cardiac output (CO) of the patient. The data (e.g., data points and/or waveforms) may be used to measure/estimate the measure mixed venous oxygen saturation of the patient. The data (e.g., data points and/or waveforms) may be used to measure/estimate the oxygen consumption (VO2), or another measure of metabolic activity, of the patient. The data (e.g., data points and/or waveforms) may be used to measure/estimate/detect low oxygen blood levels of the patient. The data (e.g., data points and/or waveforms) may be used to measure/estimate/detect retrograde blood flow of the patient. The data (e.g., data points and/or waveforms) may be used to measure/estimate/detect retrograde improper blood mixing of the patient.

In an embodiment, the system may also include one or more additional sensors. The additional sensor may be or include a temperature sensor. More particularly, the sensor may be a core body temperature sensor. For example, the sensor may be a heat flux sensor. Where the additional sensor is temperature sensor. The temperature sensor may measure changes in the body temperature, which may affect vasoconstriction/vasodilation. The additional sensor may also or instead be or include a bioimpedance sensor. The bioimpedance sensor may measure changes in fluid accumulation and/or body composition. The bioimpedance sensor may also or instead measure tidal respiratory volume. This data from the bioimpedance sensor may be used in combination with the optical signal to evaluate a parameter related to the respiratory functions of the patient. The bioimpedance sensor may also or instead be used to measure stroke volume and/or cardiac output. The data from the bioimpedance sensor may be used in combination with the optical signal to evaluate a parameter related to the cardiovascular functions of the patient.

The data from one or more of the sensors discussed above may be used in conjunction with a calorimetry device, a respiratory device, blood gas measurements, a measure of cardiac output, or a combination thereof. The data may also or instead be used as a trend monitor to measure values in reference to a baseline. The baseline may be calculated based at least partially upon the patient's demographics. The baseline may also or instead be obtained for a specific patient during examination (e.g., arterial blood gases, calorimetry tests, etc.). The baseline may also or instead be obtained from optical signal values measured when the device is first used. The baseline may also or instead be selected based at least partially upon a mathematical equation or algorithm. The equation can account for one or more of: location of placement, demographics, tests, and/or estimates.

The NIRS technique may include one or more of: time-domain diffuse optical spectroscopy (TD-NIRS), frequency-domain near-infrared spectroscopy (FD-NIRS), continuous wave near-infrared spectroscopy (CW-NIRS), near-infrared spatially resolved spectroscopy (SRS), or a combination thereof. The PPG technique may include transmittance and/or reflectance.

A system for monitoring physiological signals from a patient may include an insert configured to attach to a clothing item worn by the patient and contact a skin of the patient. The insert includes a first sensor including first and second sense electrodes configured to measure a first signal from the patient. The insert also includes a second sensor including first and second drive electrodes and the first and second sense electrodes. The first and second drive electrodes are configured to inject an electrical current into the patient, and the first and second sense electrodes are configured to measure a second signal in response to the electrical current. The system also includes a processor configured to process the first signal or a signal derived therefrom with a first algorithm to determine a heart rate of the patient, and the second signal or a signal derived therefrom with a second algorithm to determine a respiration rate of the patient. The system also includes a flexible substrate covering the first sensor and the second sensor.

The first sensor includes an ECG sensor, and the first signal includes an ECG signal. The second sensor includes a bioimpedance sensor, and the second signal includes a bioimpedance signal.

The first and second drive electrodes include bioimpedance pneumography electrodes, and the second signal includes a bioimpedance waveform from the patient.

The clothing item includes a diaper, a onesie, a waist band, a belt, one or more patches configured to adhere to the skin, or a band configured to wrap at least partially around an extremity of the patient.

The insert further includes a third sensor. The third sensor includes an oxygen saturation sensor configured to measure a third signal from the patient. The processor is configured to process the third signal or a signal derived therefrom with a third algorithm to determine an oxygen saturation in blood or a region of tissue of the patient.

The system also includes a screen configured to visually display the heart rate and the respiration rate.

The first sensor includes an optical sensor. The optical sensor includes a photodiode and a light source. The light source includes a light-emitting diode.

The first signal includes a photoplethysmogram signal. The processor is configured to process the photoplethysmogram signal or a signal derived therefrom with the first algorithm to determine the heart rate of the patient or a blood oxygenation of the patient.

The first signal includes a near-infrared spectrogram signal. The processor is configured to process the near-infrared spectrogram signal or a signal derived therefrom with the first algorithm to determine the heart rate of the patient or a regional tissue saturation of the patient.

The insert further includes a third sensor. The third sensor includes an accelerometer sensor configured to measure a third signal from the patient. The third signal includes a time-dependent waveform. The processor is configured to process the third signal or a signal derived therefrom with a third algorithm to determine a motion of the patient.

The time-dependent waveform indicates respiratory-induced motion of a torso of the patient.

The time-dependent waveform indications motion measured along an axis of the accelerometer sensor that is normal to a belly of the patient.

The insert further includes a third sensor. The third sensor includes a thermal sensor configured to measure a third signal from the patient. The processor is configured to process the third signal or a signal derived therefrom with a third algorithm to determine a core body temperature of the patient, a skin temperature of the patient, a urine temperature of the patient, a fecal temperature of the patient, or a combination thereof.

The first algorithm is a beat-picking Pan-Tompkins algorithm or a derivative thereof.

The insert further includes a third sensor configured to measure a third signal from the patient. The processor is configured to process the third signal or a signal derived therefrom with a third algorithm. The third algorithm includes a beat-picking algorithm configured to analyze bioimpedance waveforms or photoplethysmogram waveforms in the third signal to determine the heart rate.

The insert further includes a third sensor configured to measure a third signal from the patient. The processor is configured to process the third signal or a signal derived therefrom with a third algorithm. The third algorithm includes a breath-picking algorithm configured to analyze waveforms modulated by a respiration rate of the patient. The breath-picking algorithm includes a slope-summing algorithm.

The insert further includes a plurality of additional sensors configured to measure a plurality of additional signals from the patient. The processor is configured to process the additional signals or signals derived therefrom with a plurality of additional algorithms to determine a cardiac output of the patient, to determine that the patient has urinated or defecated, to determine that the patient has hyperthermia or hypothermia, to determine changes in a regional vascular resistance of the patient due to thermoregulation, or a combination thereof.

The insert further includes a plurality of additional sensors configured to measure a plurality of additional signals from the patient. The processor is configured to process the additional signals or signals derived therefrom with a plurality of additional algorithms to determine a vital sign of the patient, to determine a time-dependent physiological status of the patient, to determine a motion of the patient, to determine a posture of the patient, to determine a sleep status of the patient, to determine a urination or defecation status of the patient, or a combination thereof.

The system also includes a graphical user interface configured to display an image of the vital sign, the time-dependent physiological status, the motion, the posture, the sleep status, and the urination or defecation status.

The system also includes an alarm that is configured to be triggered in response to the vital sign being above or below a first predetermined threshold, the time-dependent physiological status being above or below a second predetermined threshold, the motion being greater than or less than a predetermined amount, the posture being in a predetermined posture state for more than a predetermined amount of time, the urination or defecation status being positive, or a combination thereof.

The processor is also configured to predict a health risk index for the patient and to predict an evolution of a health of the patient based at least partially upon the heart rate and the respiration rate.

The processor is also configured to process the first signal, the second signal, or both to determine a cardiac output of the patient, a stroke volume of the patient, a tidal volume of the patient, or a combination thereof.

### Prediction of Cardiac Index Measurement Using Non-Invasive Features

In children, cardiac index (CI) (also referred to as cardiac output) is measured invasively during a catheterization procedure. Monitoring CI non-invasively and continuously may allow earlier intervention to prevent deterioration, as well as demonstrate responsiveness to an intervention, in pediatric intensive care unit settings. The systems and methods described herein may implement a machine learning (ML) model to predict CI using non-invasive features including demographics, laboratory biomarkers, heart anatomy, and time-gated vital sign measurements.

### Patient Population

Data may include retrospective physiologic data extracted from cardiac catheterization procedures in patients with congenital heart disease (CHD) and/or following orthotopic heart transplantation. The dataset included 328 cardiac catheterization encounters from 133 unique patients with laboratory biomarkers, heart anatomy, demographics, and a comprehensive set of mathematical features extracted from vital signs. The patients included in the study ranged in age from 0 to 21 years, with an average age of 8.1 years, and 68% of the patients have a two-ventricle anatomy, while 28% have a one-ventricle anatomy.

### Outcome, Predictor Variables, and Temporal Features Extraction

CI in this analysis was obtained either by Fick or Thermodilution methods. Cardiac catheterization encounters with a CI measurement greater than 6 or less than 1.5 were excluded from the analysis (8 patient encounters). Post exclusion, the average CI value from the 320 encounters in the dataset was 3.5 L/min/m² with a standard deviation of 0.9 L/min/m².

Applicant has investigated high-frequency physiological time-series features including heart rate, systolic and diastolic blood pressure, respiratory rate, oxygen saturation, and average blood pressure which were recorded at a frequency of one measurement per four-minute time interval. The other time-series features included in the analysis were anesthesia vital signs including systolic and diastolic blood pressure, temperature, and pulse which were recorded at different frequencies, with one measurement taken per one minute and three-minute time intervals. These features have been assessed to detect hidden temporal patterns as a means of unfolding to learn the critical correlations associated with the CI measurement. For this purpose, a time window of 20 minutes was initially used. Within this window, vital signs measurements corresponding to CI encounter timestamp were extracted for analyzing temporal patterns.

A tsfresh python package was then employed to transform the characteristics of these selected measurements into an extensive collection of numerical features including descriptive summary statistics (e.g., mean, median, mode, range, variance, standard deviation, quantiles, length, kurtosis), autocorrelation (e.g., measure a relationship between a data point to a previous data point at a certain time lag), time-series quantiles (e.g., divide a time series measurement into segments such as 25^{th}, 50^{th} and 75^{th} percentiles based on their cumulative probability distribution), spectral (e.g., identify dominant frequencies and patterns in a time series measurement), Fourier (e.g., identify periodic patterns and harmonics in a time series measurement), linear (e.g., identify linear relationships estimating time series measurement trend, slope and intercept coefficients), non-linear and polynomial (e.g., capture non-linear time series behavior patterns and coefficients), wavelet (e.g., decompose time series measurement into various frequency components and capture localized changes in frequency over time) and entropy (e.g., quantifies uncertainty present in the time series measurement), etc., which may be utilized as input for a ML model. These numerical features in higher dimensions may potentially exhibit robustness against perturbations in vital sign waveforms to some degree, without significantly impacting the outcome predictions. The other predictors included in the analyses were heart anatomy, variables categorizing the CHD primary classifications, which are static for each patient and do not change with the cardiac catheterization encounters. Predictors including laboratory biomarkers and patient demographics including weight, height, and z-scores were updated with the cardiac catheterization encounters. Predictors that possess variability less than 20% in the dataset were eliminated from the analyses resulting in a sum of 1,490 potential predictors in the dataset.

### Missing Values Imputation

Patient similarity was considered when employing predictive imputation techniques to estimate missing values. An iterative imputation algorithm enabled up to 100 iterations with a decision tree regressor and was fitted to the entire dataset learning similar patient patterns and intricate relationships between the predictors. The iterative nature of the algorithm allowed for continuous refinement of the imputations, enabling the imputer to learn and adjust its prediction estimates based on the evolving understanding of the data, thereby enhancing the quality of subsequent analyses.

### Dimensionality Reduction

Following the removal of features with low variance and addressing missing values, the imputed dataset was randomly split into training (240 encounters) and testing (80 encounters) cohorts. A Boruta algorithm was applied in conjunction with the random forest regressor to the training cohort only. The objective was to determine the statistically significant features that contributed valuable information for predicting the CI.

Applicant retained only 54 contributing features for the subsequent analyses, successfully reducing the dimensionality of the dataset. This reduction in predictors may have a positive impact on improving the accuracy of CI prediction.

### Machine Learning Model Induction

The XGBoost (XGB) tree-based ML algorithm was applied to the training cohort to learn the relationships between the significant features and their corresponding CI encounter measurements. The optimization of the XGB learning process was achieved using the root mean squared error (RMSE) as a metric to quantify the model's performance. Furthermore, to fine-tune the XGB algorithm, hyperparameter tuning was conducted with 5-fold cross-validation. Bayesian optimization was also employed, and a search grid was utilized to identify the optimal XGB parameters that maximized the negative mean squared error score.

The 54 features that contributed to the outcome prediction primarily include predictors extracted from temporal patterns, which were subsequently mapped with their respective raw vital sign features. The importance of these features was determined by aggregating the weights obtained from the XGB model. The difference between the observed to predicted CI values was also calculated, and a linear regression model including 95% confidence and prediction intervals was fit to estimate the XGB model performance in the CI measurement range. All the analyses were implemented using Python version 3.9.12.

### Results

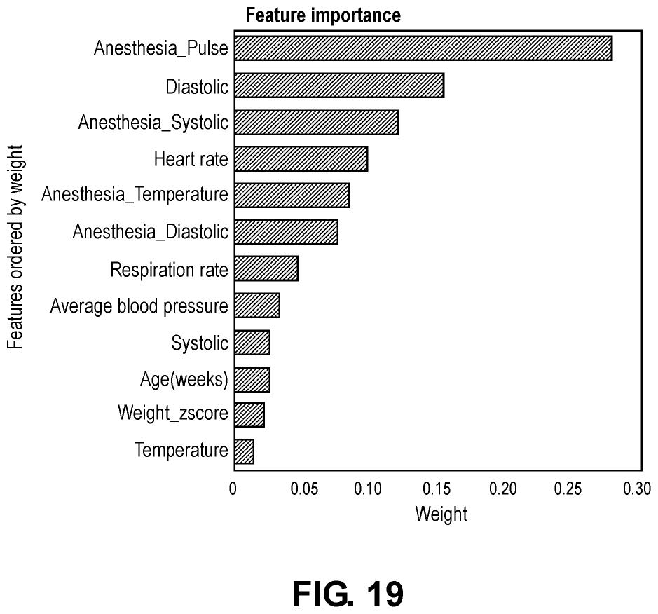

FIG. 19 illustrates a graph showing the feature importance analysis from the XGBoost model that reveals the significance of 12 variables in influencing the model's cardiac index predictions, according to an embodiment. The aggregated values of the 54 contributing predictors representing the 12 variable domains were represented with their relative contributions, with higher scores indicating greater importance.

The XGB model predicted CI with RMSE of CI±0.36 L/min/m² and CI±0.79 L/min/m² in the training and testing cohorts, respectively. The prediction model utilized 54 out of 1,490 features that were significant predictive factors as determined by the Boruta algorithm. These 54 predictors were representative of 12 variables including patient vital measurements, weight, and age. The critical vital sign measurements from anesthesia included pulse, systolic and diastolic blood pressure, temperature, and physiological measurements including heart rate, systolic and diastolic blood pressure, respiration rate, average blood pressure, and temperature. The XGB model weights for the 54 features were associated with the 12 variable domains, and their aggregation indicated the model's preference for each variable domain (FIG. 19) in predicting CI more frequently. None of the heart anatomy variables were retained as contributing predictors, potentially because the variation in measured CI explained by the anatomy information might have already been captured by other significant features present in the model.

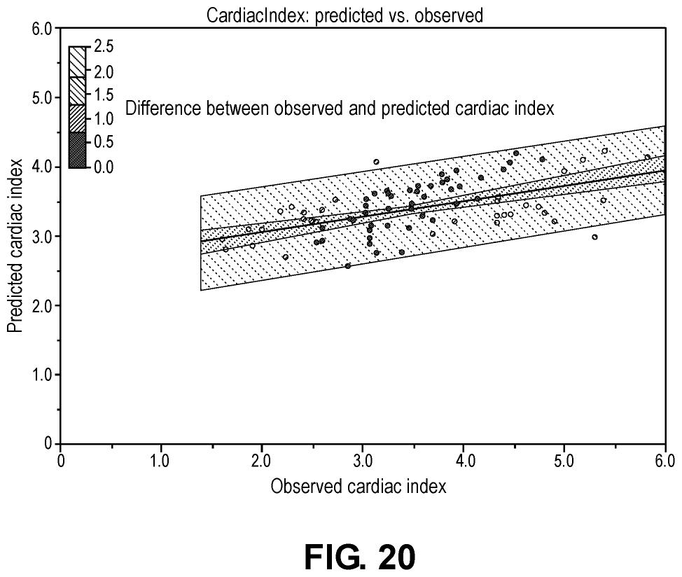

FIG. 20 illustrates a graph (e.g., scatter plot) showing a visual comparison between observed and predicted cardiac index (CI) values in the testing cohort, according to an embodiment. Each data point on the plot represents a cardiac catheterization encounter, with the observed value plotted on the x-axis and the corresponding predicted value on the y-axis. The difference between these values may be (e.g., color) coded, enabling a clear identification of the magnitude and direction of deviations between predictions and observed CI values. This informative visualization offers valuable insights into the performance and accuracy of the XGBoost prediction model, highlighting areas of agreement (denser hatching) and potential areas for improvement (less dense hatching). A linear fit regression line with a 95% confidence interval, and prediction intervals provided insights about the assessment of the XGBoost prediction model's performance, highlighting both the overall trend and variability in the CI predictions. The model exhibited with higher accuracy in the CI range of 2-4.5 L/min/m2 (RMSE<0.5), and lower accuracy in the range of <2 and >4.5 L/min/m2 (RMSE=˜1).

Thus, the model may predict CI from non-invasive, routinely collected vital signs in inpatient settings. The model may also accurately predict CI continuously and non-invasively in pediatric patients that can be implemented at the bedside.

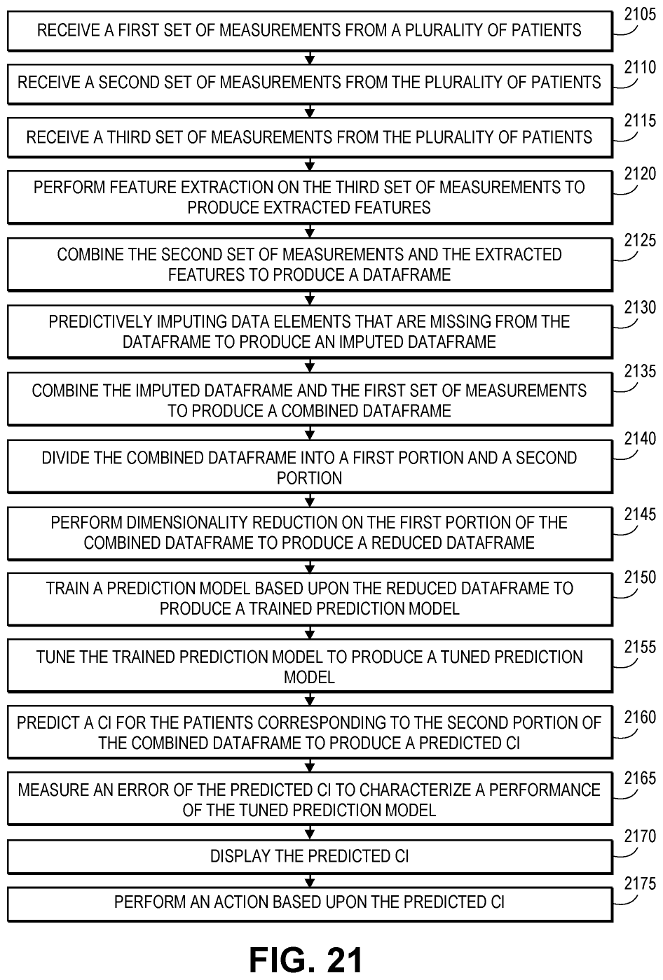

FIG. 21 illustrates a flowchart of a method 2100 for predicting a CI based upon non-invasive physiological measurements, according to an embodiment. An illustrative order of the method 2100 is provided below; however, one or more steps of the method 2100 may be performed in a different order, simultaneously, repeated, or omitted.

The method 2100 may include receiving a first set of measurements from a plurality of patients, as at 2105. The first set of measurements may include invasive physiological measurements of CI. In one embodiment, a portion of the first set of measurements that are above a first threshold and/or below a second threshold may be discarded. In an example, the first threshold is 6 ml/kg/min, and the second threshold is 1.5 ml/kg/min.

The method 2100 may also include receiving a second set of measurements from the plurality of patients, as at 2110. The second set of measurements may characterize anatomical conditions, physical conditions, and/or physiological conditions. The anatomical conditions may include a presence and a type of congenital heart malformation, a number of functional cardiac ventricles, whether any heart malformation has been repaired, a presence of residual lesions or hemodynamic abnormalities, or a combination thereof. The physical conditions may include age, gender, weight, or a combination thereof. The physiological conditions may include red blood cell count, hematocrit, or both.

The method 2100 may also include receiving a third set of measurements from the plurality of patients, as at 2115. The third set of measurements may include non-invasive physiological measurements. More particularly, the third set of measurements may include non-invasive intra-operative measurements and anesthesia vital measurements. For example, the third set of measurements may include pulse, systolic blood pressure, diastolic blood pressure, heart rate, temperature, respiratory rate, or a combination thereof.

The method 2100 may also include performing feature extraction on the third set of measurements to produce extracted features, as at 2120. Performing feature extraction may include gating the third set of measurements for a predetermined amount of time to create a time-series. Performing feature extraction may also include extracting features from the time-series to produce the extracted features. The extracted features may include descriptive summary statistics, autocorrelation, time series quantiles, spectral, Fourier, linear, non-linear, polynomial, wavelet, entropy, or a combination thereof.

The method 2100 may also include combining the second set of measurements and the extracted features to produce a dataframe, as at 2125.

The method 2100 may also include predictively imputing data elements that are missing from the dataframe to produce an imputed dataframe, as at 2130. The data elements may be predictively imputed using an iterative decision tree algorithm.

The method 2100 may also include combining the imputed dataframe and the first set of measurements to produce a combined dataframe, as at 2135.

The method 2100 may also include dividing the combined dataframe into a first portion and a second portion, as at 2140. The first portion may be used for training a predictive model, and wherein the second portion is used for testing and/or tuning the predictive model.

The method 2100 may also include performing dimensionality reduction on the first portion of the combined dataframe to produce a reduced dataframe, as at 2145. Performing the dimensionality reduction removes a subset of the data elements from the first portion of the combined dataframe to produce the reduced dataframe. The dimensionality reduction may be performed using a random forest tree regressor algorithm.

The method 2100 may also include training a prediction model based upon the reduced dataframe to produce a trained prediction model, as at 2150. The prediction model may be trained using an XGBoost tree algorithm.

The method 2100 may also include tuning the trained prediction model to produce a tuned prediction model, as at 2155. Tuning the trained prediction model may modify (e.g., optimize) hyperparameters using a Bayesian search technique by applying a 5-fold cross-validation.

The method 2100 may also include predicting the CI for the patients corresponding to the second portion of the combined dataframe to produce a predicted CI, as at 2160. The CI may be predicted using the tuned prediction model and based at least partially upon the dataframe.

The method 2100 may also include measuring an error of the predicted CI to characterize a performance of the tuned prediction model, as at 2165. This may be done by comparing the predicted CI to the second set of measurements. The error may be measured by calculating a root mean square error (RMSE) metric of the predicted CI versus the first set of measurements, the second set of measurements, the third set of measurements, or a combination thereof.

The method 2100 may also include displaying the predicted CI, as at 2170.

The method 2100 may also include performing an action based upon the predicted CI, as at 2175. The action may be or include capturing additional invasive physiological measurements from the plurality of patients, changing medications of the plurality of patients, performing a surgical intervention on the plurality of patients, or a combination thereof.

1. A method for predicting a cardiac index (CI) for a patient, the method comprising:
receiving a set of a measurements from the patient that characterizes an anatomical condition, a physical condition, a physiological condition, or a combination thereof;
receiving a non-invasive physiological measurement from the patient;
performing feature extraction on the non-invasive physiological measurement to produce one or more extracted features;
combining the set of measurements and the extracted features to produce a dataframe; and
predicting the CI for the patient to produce a predicted CI, wherein the CI is predicted using a prediction model based at least partially upon the dataframe.

2. The method of claim 1, wherein the set of measurements comprises the anatomical condition, and wherein the anatomical condition comprises a presence and a type of congenital heart malformation, a number of functional cardiac ventricles, whether any heart malformation has been repaired, a presence of residual lesions or hemodynamic abnormalities, or a combination thereof.

3. The method of claim 1, wherein the set of measurements comprises the physical condition, and wherein the physical condition comprises age, gender, weight, or a combination thereof.

4. The method of claim 1, wherein the set of measurements comprises the physiological condition, and wherein the physiological condition comprises a red blood cell count, a hematocrit, or both.

5. The method of claim 1, wherein the non-invasive physiological measurement comprises a non-invasive intra-operative measurement, an anesthesia vital measurement, or both.

6. The method of claim 1, wherein the non-invasive physiological measurement comprises a pulse, a systolic blood pressure, a diastolic blood pressure, a heart rate, a temperature, a respiratory rate, or a combination thereof.

7. The method of claim 1, wherein the performing feature extraction comprises:
gating the non-invasive physiological measurement for a predetermined amount of time to create a time-series; and
extracting features from the time-series to produce the one or more extracted features.

8. The method of claim 1, wherein the one or more extracted features comprise descriptive summary statistics, autocorrelation, time series quantiles, spectral, Fourier, linear, non-linear, polynomial, wavelet, entropy, or a combination thereof.

9. The method of claim 1, further comprising displaying the predicted CI.

10. The method of claim 1, further comprising performing an action based upon the predicted CI, wherein the action comprises capturing an invasive physiological measurement from the patient, changing a medication for the patient, performing a surgical intervention on the patient, or a combination thereof.

11. A method for predicting cardiac indexes (CIs) for a plurality of patients based upon non-invasive physiological measurements from the patients, the method comprising:
receiving a first set of measurements from the plurality of patients, wherein the first set of measurements comprises invasive physiological measurements of CI;
receiving a second set of measurements from the plurality of patients, wherein the second set of measurements characterize anatomical conditions, physical conditions, physiological conditions, or a combination thereof;
receiving a third set of measurements from the plurality of patients, wherein the third set of measurements comprises non-invasive physiological measurements;
performing feature extraction on the third set of measurements to produce extracted features;
combining the second set of measurements and the extracted features to produce a dataframe;
training a prediction model based at least partially upon the dataframe to produce a trained prediction model; and
predicting the CIs for at least a portion of the patients using the trained prediction model based at least partially upon the dataframe.

12. The method of claim 11, wherein the anatomical conditions comprise a presence and a type of congenital heart malformation, a number of functional cardiac ventricles, whether any heart malformation has been repaired, a presence of residual lesions or hemodynamic abnormalities, or a combination thereof, wherein the physical conditions comprise age, gender, weight, or a combination thereof, and wherein the physiological conditions comprise red blood cell count, hematocrit, or both.

13. The method of claim 11, wherein the third set of measurements comprises pulse, systolic blood pressure, diastolic blood pressure, heart rate, temperature, respiratory rate, or a combination thereof.

14. The method of claim 11, wherein the performing feature extraction comprises:
gating the third set of measurements for a predetermined amount of time to create a time-series; and
extracting features from the time-series to produce the extracted features, wherein the extracted features comprise descriptive summary statistics, autocorrelation, time series quantiles, spectral, Fourier, linear, non-linear, polynomial, wavelet, entropy, or a combination thereof.

15. The method of claim 11, further comprising:
predictively imputing data elements that are missing from the dataframe to produce an imputed dataframe;
combining the first set of measurements and the imputed dataframe to produce a combined dataframe;
dividing the combined dataframe into a first portion and a second portion; and
performing dimensionality reduction on the first portion of the combined dataframe to produce a reduced dataframe, wherein the prediction model is trained based upon the reduced dataframe, and wherein the CI is predicted for the patients corresponding to the second portion of the combined dataframe to produce the predicted CI.

16. A method for predicting cardiac indexes (CIs) for a plurality of patients based upon non-invasive physiological measurements from the patients, the method comprising:
receiving a first set of measurements from the plurality of patients, wherein the first set of measurements comprises invasive physiological measurements of CI;
receiving a second set of measurements from the plurality of patients, wherein the second set of measurements characterize anatomical conditions, physical conditions, and physiological conditions, wherein the anatomical conditions comprise a presence and a type of congenital heart malformation, a number of functional cardiac ventricles, whether any heart malformation has been repaired, a presence of residual lesions or hemodynamic abnormalities, or a combination thereof, wherein the physical conditions comprise age, gender, weight, or a combination thereof, and wherein the physiological conditions comprise red blood cell count, hematocrit, or both;
receiving a third set of measurements from the plurality of patients, wherein the third set of measurements comprises non-invasive physiological measurements, wherein the third set of measurements comprises non-invasive intra-operative measurements and anesthesia vital measurements, wherein the third set of measurements comprises pulse, systolic blood pressure, diastolic blood pressure, heart rate, temperature, respiratory rate, or a combination thereof;
performing feature extraction on the third set of measurements to produce extracted features, wherein the performing feature extraction comprises:
gating the third set of measurements for a predetermined amount of time to create a time-series; and
extracting features from the time-series to produce the extracted features, wherein the extracted features comprise descriptive summary statistics, autocorrelation, time series quantiles, spectral, Fourier, linear, non-linear, polynomial, wavelet, entropy, or a combination thereof;
combining the second set of measurements and the extracted features to produce a dataframe;
predictively imputing data elements that are missing from the dataframe to produce an imputed dataframe;
combining the first set of measurements and the imputed dataframe to produce a combined dataframe;
dividing the combined dataframe into a first portion and a second portion;
performing dimensionality reduction on the first portion of the combined dataframe to produce a reduced dataframe, wherein performing the dimensionality reduction removes a subset of the data elements from the first portion of the combined dataframe to produce the reduced dataframe, and wherein the dimensionality reduction is performed using a random forest tree regressor algorithm;
training a prediction model based upon the reduced dataframe to produce a trained prediction model, wherein the prediction model is trained using an XGBoost tree algorithm;
tuning the trained prediction model to produce a tuned prediction model, wherein tuning the trained prediction model optimizes hyperparameters using a Bayesian search technique by applying a 5-fold cross-validation; and
predicting the CIs for the patients corresponding to the second portion of the combined dataframe to produce predicted CIs, wherein the CIs are predicted using the tuned prediction model.

17. The method of claim 16, further comprising measuring an error of the predicted CIs to characterize a performance of the tuned prediction model.

18. The method of claim 17, wherein the error is measured by calculating a root mean square error (RMSE) metric of the predicted CIs versus the second set of measurements.

19. The method of claim 16, further comprising displaying the predicted CIs.

20. The method of claim 16, further comprising performing an action based upon the predicted CIs, wherein the action comprises capturing additional invasive physiological measurements, changing a medication, performing a surgical intervention, or a combination thereof.
